(19) United States
(12) Patent Application Publication
(10) Pub. No.: US 2025/0205406 A1
(43) Pub. Date: Jun. 26, 2025

(54) BIDIRECTIONAL FLOW CANNULAS, SYSTEMS INCLUDING BIDIRECTIONAL CANNULAS, AND METHODS OF USING SAME

(71) Applicant: Total Flow Medical Limited, Vancouver (CA)

(72) Inventors: Hillary Pierce, Vancouver (CA); Martina Wan Lockwood, Vancouver (CA)

(21) Appl. No.: 18/846,640

(22) PCT Filed: Mar. 15, 2023
(86) PCT No.: PCT/IB2023/052484

§ 371 Date: Sep. 12, 2024

Related U.S. Application Data
(60) Provisional application No. 63/320,174, filed on Mar. 15, 2022.

(51) Int. Cl. A61M 1/36; A61M 25/00; A61M 25/10

(52) U.S. Cl. CPC A61M 1/3659; A61M 1/3666; A61M 25/0032; A61M 25/007; A61M 25/1002; A61M 2025/0031; A61M 2025/1052; A61M 2025/1079

(57) ABSTRACT

Bidirectional flow cannulas may include a main cannula tube with a central lumen that is configured to be in liquid communication with a source of blood once a portion of the bidirectional flow cannula is positioned within a patient's blood vessel. The bidirectional flow cannula may include an inflatable balloon that circumferentially surrounds a distally positioned portion of an exterior diameter the main tube that is configured to be inflated and/or deflated via an inflation line coupled to the main cannula tube. The bidirectional flow cannula may further include a first plurality of ports positioned within the main cannula tube on a proximal side of the inflatable balloon and a second plurality of ports positioned within the main cannula tube on a distal side of the inflatable balloon, and, on some occasions, the first plurality of ports including more ports than the second plurality of ports.

### RELATED APPLICATION

This application is an international patent application of, and claims priority to, U.S. Provisional Patent Application No. 63/320,174 filed 15 Mar. 2022 and entitled “BIDIRECTIONAL FLOW CANNULAS, SYSTEMS INCLUDING BIDIRECTIONAL CANNULAS, AND METHODS OF USING SAME,” which is incorporated herein in its entirety.

### INVENTIVE FIELD

The invention pertains to bidirectional flow cannulas for use within vessels in the body, such as blood vessels in a limb, that are configured to provided blood flow in two directions to both the body and head as well as the cannulated limb.

### BACKGROUND

Femoral cannulation during, for example, minimally invasive cardiac surgery or extracorporeal membrane oxygenation (ECMO) is often fraught with a host of complications ranging in severity from bleeding to sepsis to death. For example, the presence of a femoral arterial and venous cannula to achieve adequate cardiac output can significantly impair blood flow and venous drainage from the leg, which may lead to ischemic conditions of the cannulated limb and/or reperfusion injuries following decannulation. This presents numerous challenges to the clinician. If not identified early, the consequences of limb ischemia are numerous, often requiring surgical intervention and challenges with blood pressure and hemodynamic patient management. Additional consequences of limb ischemia are significant. Venous thrombosis is a common morbidity, while tissue necrosis would be the ultimate sequelae. Further interventions are routinely required in the presence of limb ischemia such as embolectomies, fasciotomies, and amputations.

Minimally invasive cardiac surgery (MICS) represents a safe and effective approach for a variety of cardiac surgical diseases. The use of minimally invasive cardiac surgery has continued to increase over the last 10-15 years for the aging population with the number of patients undergoing reoperation for valvular heart disease increasing as the general population ages and has demonstrated better long-term survival in the elderly.

During MICS, the use of peripheral cannulation reduces the chest incision and maximizes the operative space. Femoral cannulation is commonly used for patients undergoing cardiopulmonary bypass in approximately 85-90% of MICS and 20% of all cardiac surgeries. To perform this peripheral cannulation, femoral cannulas are placed into the femoral artery and vein by a cardiac surgeon in preparation for initiating cardiopulmonary bypass. These cannulas remove and return the patient's blood to and from the cardiopulmonary bypass machine providing extracorporeal circulation during surgery.

One of the major risks associated with traditional femoral cannulation is limb ischemia. Limb ischemia occurs as a result of a lack of blood flow in a patient's leg and is associated with increased morbidity and mortality for patients undergoing extracorporeal circulation using femoral cannulation. There are additional factors which may increase the likelihood of developing ischemic limb conditions by affecting perfusion to the ipsilateral leg. These include the use of high vasopressor support and the use of large caliber flow-occlusive systemic femoral cannula.

It is hypothesized that ischemic limb conditions during traditional femoral cannulation are caused by fluid mechanics (e.g., the Bernoulli principle) that a lack of blood flow to the ipsilateral limb because a higher velocity blood flow exiting the tip of the conventional cannula in the direction of the head and body creates negative pressure in the femoral artery below the tip of the conventional cannula, effectively pulling blood up the artery from the leg, in the direction of the body. This retrograde blood flow is a cause of ischemic limb conditions.

### SUMMARY

The bidirectional flow cannulas disclosed herein may be configured to reduce and/or eliminate ischemic conditions within a cannulated limb (e.g., leg) and/or reperfusion injuries by, for example, directing blood flow to the ipsilateral cannulated limb and/or providing one or more mechanisms (e.g., ports) for the delivery of blood to a cannulated blood vessel, which may serve to reduce a velocity and/or pressure of the blood being introduced into the vessel via the bidirectional flow cannula, which may reduce a likelihood of hemolysis for the patient.

In many cases, the bidirectional flow cannulas disclosed herein are configured for insertion into a blood vessel (e.g., femoral artery or femoral vein) of a patient's limb (e.g., leg or arm) so that blood may be supplied to the patient during, for example, the use of extracorporeal membrane oxygenation (ECMO) and/or during heart surgery utilizing cardiopulmonary bypass (CPB). At times, the bidirectional flow cannulas disclosed herein may include a radio-opaque marker configured to assist with the insertion of the bidirectional flow cannula. The radio-opaque marker may be positioned on the distal side of the main cannula tube. In many circumstances, the bidirectional flow cannulas disclosed herein may be configured so that they may be inserted into the femoral artery and moved into the external iliac artery or common iliac artery where the bidirectional flow cannula, or a portion thereof, may reside during use and/or until removal. Guiding the bidirectional flow cannula through the femoral artery to the external iliac artery or common iliac artery may be performed with the assistance of one or more imaging and/or visualization techniques (e.g., ultrasound and/or X-ray) that may, for example show a position of the radio-opaque marker in the patient's femoral, external iliac, or common iliac artery.

The bidirectional flow cannulas disclosed herein may include a main cannula tube with a central lumen that has an open end through which fluids, such as blood, may flow into the patient's blood vessel toward the patient's heart, head, and/or body during, for example, ECMO and/or support during cardiopulmonary bypass. The flow of blood through the central lumen and out of the open end may be referred to herein as “a primary blood flow.” The main cannula tube may be configured to be coupled to, and in fluid communication with, a source of blood and/or fluid via, for example, a coupling and/or another tube connected to the main cannula tube that includes a coupling. The bidirectional flow cannula may further include a vessel occlusion device such as an inflatable balloon that circumferentially surrounds a distally positioned portion of an exterior diameter the main tube. The vessel occlusion device may be inflatable and/or deflatable via communication with an inflation line that may be configured to facilitate inflation/deflation of the occlusion device/inflatable balloon via a coupling to, for example, a supply of air, gas, liquid (e.g., saline), and/or negative pressure that may be provided by, for example, an air, liquid, and/or vacuum pump. In some embodiments, the inflatable balloon may be configured so that it does not fully occlude a patient's blood vessel when positioned therein and inflated. Additionally, or alternatively, the inflatable balloon may have a circular or non-circular cross-section, the cross-section being perpendicular to the main cannula tube.

The bidirectional flow cannula may also include a first plurality of ports (e.g., 1-18) positioned within the main cannula tube on a proximal side of the inflatable balloon. The first plurality of ports may be configured, positioned, and/or arranged within main cannula tube to facilitate a secondary flow of blood from the main lumen of the main cannula tube to flow through the first plurality of ports. This secondary blood flow may travel in a retrograde direction (i.e., in a direction opposite to the primary blood flow) toward the patient's limb thereby providing oxygenated blood to the patient's limb, which may prevent ischemia of the patient's limb.

Optionally, the bidirectional flow cannula may include a second plurality (e.g., 2-16) of ports positioned within the main cannula tube on a distal side of the inflatable balloon, the second plurality of ports may include more ports than the first plurality of ports. The second plurality of ports may be configured, positioned, and/or arranged within main cannula tube to facilitate a secondary pathway for blood to enter the patient's blood vessel with this blood traveling in the same direction as the primary blood flow exiting the open end of the central lumen. Blood flowing through the second plurality of ports may serve to, for example, decrease a pressure and/or a velocity of blood flowing exiting the open end of the central lumen while still providing enough blood to the vessel for communication to the patient's heart and body, which may, for example, mitigate injury to the vessel caused by blood entering the vessel under high pressure and/or at a relatively high velocity.

In some embodiments, an interior diameter of the second plurality of ports may be smaller than an interior diameter of the first plurality of ports. Alternatively, an interior diameter of the first plurality of ports may be smaller than an interior diameter of the second plurality of ports.

In some embodiments, a port of at least one of the first plurality of ports and the second plurality of ports may be oriented at an angle that may be not perpendicular to the main cannula tube. Additionally, or alternatively, a quantity (e.g., 1-14) of ports included in the first plurality of ports may be larger than a quantity of ports included in the second plurality of ports. For example, in one embodiment, the first plurality of ports may include eight ports and the second plurality of ports may include two ports. Additionally, or alternatively, wherein a quantity (e.g., 1-14) of ports included in the second plurality of ports may be larger than a quantity of ports included in the first plurality of ports.

In some embodiments, the bidirectional flow cannulas disclosed herein may include a proximal basket positioned within a sidewall of the proximal side of the main cannula tube. The proximal basket may include a first plurality of holes sized, positioned, and configured, to align with the first plurality of ports. Additionally, or alternatively, the bidirectional flow cannulas disclosed herein may include a distal basket positioned within a sidewall of the distal side of the main cannula tube. The distal basket may include a second plurality of holes sized, positioned, and configured, to align with the second plurality of ports.

In some embodiments, the inflation line may have a non-circular cross-section, the cross-section being perpendicular to the main cannula tube. Additionally, or alternatively, the inflation line may be positioned on an exterior surface of the main cannula tube and the bidirectional flow cannula also include a set of supports positioned at a junction of the inflation line and the main cannula tube. Additionally, or alternatively, the inflation line may be positioned within a sidewall of the main cannula tube.

In some instances, the bidirectional flow cannulas disclosed herein may be used by inserting a distal portion of the bidirectional flow cannula into a blood vessel in a patient's limb, wherein the distal portion of the bidirectional flow cannula may include an inflatable balloon. Once in situ in the patient's blood vessel, the inflatable balloon may be inflated to a volume that partially occludes the patient's blood vessel. In some embodiments, the inflatable balloon may be inflated to a predetermined level of pressure and/or a predetermined volume of gas or liquid that is calculated to partially inflation the balloon and/or inflate the balloon to a volume that only partially occludes the patient's blood vessel. In some cases, an indication of a size of the blood vessel may be received prior to insertion of the bidirectional flow cannula into the bidirectional flow cannula and a volume of gas provided to the inflatable balloon and/or a pressure within the inflatable balloon may be responsive to the received indication. In some instances, when a certain level of back pressure is measured when inflating the inflatable balloon, it may be deduced that the inflatable balloon is fully occluding the blood vessel (because the blood vessel is exerting an opposite force on the balloon, thereby increasing the air pressure within the balloon). When this level of back pressure is received, the balloon may be deflated by an amount sufficient to reduce its volume so that it does not fully occlude the vessel.

Once the bidirectional flow cannula is positioned within the patient's vessel, a volume of blood may be supplied to the bidirectional flow cannula so that the volume of blood may be injected into the patient's blood vessel via a tip of the bidirectional flow cannula positioned on a distal end of the bidirectional flow cannula, wherein a first portion of the volume of blood supplied to the bidirectional flow cannula flows away from the bidirectional flow cannula along the patient's blood vessel toward the patient's heart and a second portion of the volume of blood supplied to the bidirectional flow cannula flows toward, and past, the inflatable balloon toward the patient's canulated limb.

In some embodiments, the bidirectional flow cannula may further include a plurality of ports positioned on a proximal side of the inflatable balloon, wherein a third portion of the volume of blood supplied to the bidirectional flow cannula flows from the ports and toward the patient's limb. Additionally, or alternatively, the bidirectional flow cannula may further include a plurality of ports positioned on a distal side of the inflatable balloon, wherein a fourth portion of the volume of blood supplied to the bidirectional flow cannula flows from the ports positioned on a distal side of the inflatable balloon and toward the patient's heart, thereby reducing at least one of a velocity and a pressure of the first portion of blood exiting the tip of the bidirectional flow cannula.

Additionally, or alternatively, use of the bidirectional flow cannulas disclosed herein may comprise inserting a distal portion of the bidirectional flow cannula into a blood vessel in a patient's limb, the distal portion of the bidirectional flow cannula may include an inflatable balloon and a plurality of ports positioned on a proximal side of the inflatable balloon. Then, once in situ in the patient's blood vessel, the inflatable balloon may be inflated to a volume that fully or partially occludes the patient's blood vessel and a volume of blood may be supplied to the bidirectional flow cannula so that the volume of blood may be injected into the patient's blood vessel via a tip of the bidirectional flow cannula positioned on a distal end of the bidirectional flow cannula, wherein a first portion of the volume of blood supplied to the bidirectional flow cannula flows away from the bidirectional flow cannula along the patient's blood vessel toward the patient's heart and a second portion of the volume of blood supplied to the bidirectional flow cannula flows from the plurality of ports toward the patient's limb.

### BRIEF DESCRIPTION OF THE DRAWINGS

The present invention is illustrated by way of example, and not limitation, in the figures of the accompanying drawings in which:

FIG. 1A is a side view of an exemplary bidirectional flow cannula, in accordance with some embodiments of the present invention.

FIG. 1B is a top-perspective view of an exemplary proximal basket included in the bidirectional flow cannula of FIG. 1A, in accordance with some embodiments of the present invention.

FIG. 1C is a top-perspective view of an exemplary distal basket included in the bidirectional flow cannula of FIG. 1A, in accordance with some embodiments of the present invention.

FIG. 1D is a close up view of a distal portion of the bidirectional flow cannula of FIG. 1A without a distal or proximal basket, in accordance with some embodiments of the present invention.

FIG. 1E is a close up view of a distal portion of the bidirectional flow cannula of FIG. 1A, in accordance with some embodiments of the present invention.

FIG. 2A provides a side view of a distal portion of an exemplary bidirectional flow cylindrically shaped balloon, in accordance with some embodiments of the present invention.

FIG. 2B provides a side view of a distal portion of an exemplary bidirectional flow cannula with an oval-shaped balloon, in accordance with some embodiments of the present invention.

FIG. 2C provides a side view of a distal portion of an exemplary bidirectional flow cannula that includes a rounded square-shaped balloon, in accordance with some embodiments of the present invention.

FIG. 2D provides a side view of a distal portion of an exemplary bidirectional flow cannula that includes a triangularly shaped balloon, in accordance with some embodiments of the present invention.

FIG. 2E provides a side view of a distal portion of an exemplary bidirectional flow cannula that includes a spherically shaped balloon, in accordance with some embodiments of the present invention.

FIG. 2F provides a side view of a distal portion of an exemplary bidirectional flow cannula that includes a rhomboid-shaped balloon, in accordance with some embodiments of the present invention.

FIG. 2G provides a cross section of the bidirectional flow cannulas as shown in FIGS. 2A, 2B, and 2E, in accordance with some embodiments of the present invention.

FIG. 2H shows a cross-section of an exemplary bidirectional flow cannula with a square-shaped balloon that surrounds the main cannula tube, in accordance with some embodiments of the present invention.

FIG. 2I shows a cross-section of an exemplary bidirectional flow cannula with a hexagonally shaped balloon that surrounds the main cannula tube, in accordance with some embodiments of the present invention.

FIG. 2J shows a cross-section of an exemplary bidirectional flow cannula with a substantially ovoid-shaped balloon that surrounds the main cannula tube, in accordance with some embodiments of the present invention.

FIG. 2K shows a cross-section of an exemplary bidirectional flow cannula with a substantially pentagonal-shaped balloon that surrounds the main cannula tube, in accordance with some embodiments of the present invention.

FIG. 2L shows a cross-section of an exemplary bidirectional flow cannula with a substantially rhomboid-shaped balloon that surrounds the main cannula tube, in accordance with some embodiments of the present invention.

FIG. 2M shows a cross-section of an exemplary bidirectional flow cannula with a substantially rounded-rectangularly shaped balloon that surrounds the main cannula tube, in accordance with some embodiments of the present invention.

FIG. 2N shows a cross-section of an exemplary bidirectional flow cannula with a substantially rounded square shaped balloon that surrounds the main cannula tube, in accordance with some embodiments of the present invention.

FIG. 2O shows a cross-section of an exemplary bidirectional flow cannula with a substantially triangularly shaped balloon that surrounds the main cannula tube, in accordance with some embodiments of the present invention.

FIG. 3A provides a vertical cross-section view of the bidirectional flow cannula of FIG. 1A, in accordance with some embodiments of the present invention.

FIG. 3B provides a vertical cross-section view an exemplary bidirectional flow cannula that includes an inflation line lumen resident within a main cannula tube of the bidirectional flow cannula, in accordance with some embodiments of the present invention.

FIG. 3C provides a vertical cross-section view a cross-section view of another exemplary bidirectional flow cannula that includes an inflation line lumen resident within a main cannula tube of the bidirectional flow cannula, in accordance with some embodiments of the present invention.

FIG. 3D provides a vertical cross-section view of an exemplary bidirectional flow cannula with a crescent-shaped inflation line lumen resident within a sidewall of a main cannula tube of the bidirectional flow cannula, in accordance with some embodiments of the present invention.

FIG. 3E provides a vertical cross-section view of an exemplary bidirectional flow cannula that includes an oval-shaped shaped inflation line lumen positioned within a sidewall of the main cannula tube of the bidirectional flow cannula, in accordance with some embodiments of the present invention.

FIG. 3F provides a vertical cross-section view of an exemplary bidirectional flow cannula that includes an oval-shaped inflation line, in accordance with some embodiments of the present invention.

FIG. 3G provides a vertical cross-section view of an exemplary bidirectional flow cannula that includes an oval-shaped inflation line with a first type of supports for the inflation line, in accordance with some embodiments of the present invention.

FIG. 3H provides a vertical cross-section view of an exemplary bidirectional flow cannula that includes an oval-shaped inflation line with a second type of supports for the inflation line, in accordance with some embodiments of the present invention.

FIG. 3I provides a vertical cross-section view of an exemplary bidirectional flow cannula that includes a circular-shaped inflation line with supports for the inflation line, in accordance with some embodiments of the present invention.

FIG. 3J provides a vertical cross-section view of an exemplary bidirectional flow cannula that includes a crescent shaped inflation line, in accordance with some embodiments of the present invention.

FIG. 4A1 provides a top view of a portion of a first region and/or second region of an exemplary bidirectional flow cannula that includes a port with an approximately circular shape, in accordance with some embodiments of the present invention.

FIG. 4A2 is a vertical cross-section view of the bidirectional flow cannula of FIG. 4A1, in accordance with some embodiments of the present invention.

FIG. 4B is a top view of a first region and/or a second region of a bidirectional flow cannula that includes an oval-shaped port, in accordance with some embodiments of the present invention.

FIG. 4C is a top view of a first region and/or a second region of a bidirectional flow cannula that includes a triangularly shaped port, in accordance with some embodiments of the present invention.

FIG. 4D is a top view of a first region and/or a second region of a bidirectional flow cannula that includes a square-shaped port, in accordance with some embodiments of the present invention.

FIG. 4E is a top view of a first region and/or a second region of a bidirectional flow cannula that includes an array of two ports arranged in a diagonal and linear fashion, in accordance with some embodiments of the present invention.

FIG. 4F is a top view of a first region and/or a second region of a bidirectional flow cannula that includes an array of three ports arranged in a triangular fashion, in accordance with some embodiments of the present invention.

FIG. 4G is a top view of a first region and/or a second region of a bidirectional flow cannula that includes an array of three ports arranged in a linear fashion, in accordance with some embodiments of the present invention.

FIG. 4H is a top view of a first region and/or a second region of a bidirectional flow cannula that includes an array of five ports arranged in a “X”-like fashion, in accordance with some embodiments of the present invention.

FIG. 5A provides a cross-section view of a portion of an exemplary main cannula tube with a first pair of ports, in accordance with some embodiments of the present invention.

FIG. 5B depicts a cross-section view of a portion of an exemplary main cannula tube with a second pair of ports, in accordance with some embodiments of the present invention.

FIG. 5C is a cross-section view of a portion of an exemplary main cannula tube with a third pair of ports, in accordance with some embodiments of the present invention.

FIG. 5D is a cross-section view of a portion of an exemplary main cannula tube with a fourth pair of ports, in accordance with some embodiments of the present invention.

FIG. 5E is a cross-section view of a portion of an exemplary main cannula tube with a fifth pair of ports, in accordance with some embodiments of the present invention.

FIG. 6 provides a side view of a bidirectional flow cannula positioned within a patient's blood vessel, in accordance with some embodiments of the present invention.

FIG. 7A is a side view of an introducer, in accordance with some embodiments of the present invention.

FIG. 7B provides a horizontal cross-section view of a first introducer/canula system that includes a bidirectional flow cannula with a tapered tip and an introducer, in accordance with some embodiments of the present invention.

FIG. 7C provides a cross-section view of a second introducer/canula system that includes a bidirectional flow cannula with a narrowed tip and an introducer, in accordance with some embodiments of the present invention.

FIG. 7D provides a cross-section view of a third introducer/canula system that includes a bidirectional flow cannula with a tapered and narrowed tip and an introducer, in accordance with some embodiments of the present invention.

FIG. 7E1 provides a cross-section view of a fourth introducer/canula system that includes a bidirectional flow cannula and an introducer with a feedback mechanism positioned within blood vessel, in accordance with some embodiments of the present invention.

FIG. 7E2 provides a cross-section view of the introducer with feedback mechanism of FIG. 7E1, in accordance with some embodiments of the present invention.

FIG. 7E3 is a cross section view of the introducer with feedback mechanism of FIG. 7E1, in accordance with some embodiments of the present invention.

FIG. 7F provides a cross-section view of a fifth introducer/canula system that includes a bidirectional flow cannula and an introducer with a feedback mechanism in the form of tapered proximal end, in accordance with some embodiments of the present invention.

FIG. 7G provides a cross-section view of a sixth introducer/canula system 716 that includes a bidirectional flow cannula and an introducer, in accordance with some embodiments of the present invention.

FIG. 8A is an illustration of a system for mitigating bleeding from a bidirectional flow cannula insertion site that includes a bidirectional flow cannula, in accordance with some embodiments of the present invention.

FIG. 8B1 is an illustration of an exemplary bidirectional flow cannula that includes a bleeding mitigation mechanism, in accordance with some embodiments of the present invention.

FIG. 8B2 is an illustration of the bidirectional flow cannula of FIG. 8B1 positioned within a patient's blood vessel with the bleeding mitigation mechanism deployed so it occludes an opening in the blood vessel, in accordance with some embodiments of the present invention.

FIG. 9A is a side view of a first skirted bidirectional flow canula positioned within a blood vessel, in accordance with some embodiments of the present invention.

FIG. 9B is a side view of a second skirted bidirectional flow canula positioned within a blood vessel, in accordance with some embodiments of the present invention.

FIG. 9C is a side view of a third skirted bidirectional flow canula positioned within a blood vessel, in accordance with some embodiments of the present invention.

FIG. 10 provides a flowchart of an exemplary process for setting parameters for inflation of balloon of a bidirectional flow cannula so that the balloon partially occludes a vessel into which the bidirectional flow cannula is inserted, in accordance with some embodiments of the present invention.

FIG. 11 provides a flowchart of another exemplary process for setting parameters for inflation of balloon of a bidirectional flow cannula so that the balloon partially occludes a vessel into which the bidirectional flow cannula is inserted, in accordance with some embodiments of the present invention.

FIG. 12A shows an exemplary bidirectional flow cannula positioned with a patient's blood vessel with balloon completely deflated, in accordance with some embodiments of the present invention.

FIG. 12B shows the bidirectional flow cannula of FIG. 12A positioned with a patient's blood vessel with the balloon inflated so that it partially occludes the patient's blood vessel, in accordance with some embodiments of the present invention.

FIG. 12C is a cross section of the patient's blood vessel when it is partially occluded by the balloon as shown in FIG. 12B, in accordance with some embodiments of the present invention.

FIG. 12D shows the bidirectional flow cannula of FIG. 12A positioned with a patient's blood vessel with balloon inflated and occluding the patient's blood vessel, in accordance with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

The bidirectional flow cannulas disclosed herein may be used to cannulate blood vessels and connect with other extracorporeal circulatory equipment for patients requiring arterial femoral cannulation for extracorporeal circulation during, for example, cardiopulmonary bypass procedures. The bidirectional flow cannulas disclosed herein may be used to cannulate vessels and connect with accessory extracorporeal equipment. Insertion and placement of a bidirectional flow cannula within a blood vessel may be facilitated via a cannula introducer that resides in a main lumen of the bidirectional flow cannula during the insertion procedure. The introducer may be configured to add stiffness to the bidirectional flow cannula and/or provide a handle by which to manipulate and control movement of the bidirectional flow cannula during placement within the vessel. When the bidirectional flow cannula is placed within the vessel, the introducer may be extracted from the main lumen and the bidirectional flow cannula may be coupled to a source of extracorporeal circulation during, for example, performance of cardiopulmonary bypass.

The bidirectional flow cannulas disclosed herein are configured and designed to, for example, replace conventional femoral arterial cannulas for extracorporeal circulation during, for example, cardiopulmonary bypass procedures and are advantageous because they provide, for example, an optional dedicated blood flow to the ipsilateral limb. Dedicated blood flow to the limb can be employed as a preventative measure or activated in response to a decrease in blood flow to the limb, based on the clinician's medical judgment.

In some embodiments, the bidirectional flow cannulas disclosed herein may deliver retrograde systemic blood flow to the patient's head and body while a balloon thereof is deflated and/or inflated. With the inflation of a bidirectional flow cannula balloon to the maximum diameter, blood flow may be directed to the patient's head and body and directed to the patient's leg via one or more ports located through a main lumen of the bidirectional flow cannula (carrying oxygenated blood).

The blood flow ports (also called “ports” herein) may be located on proximal and/or distal sides of the balloon. The balloon may be configured and/or positioned on the bidirectional flow cannula so that it separates the proximal blood flow ports from distal proximal ports along the insertable length of the cannula, which may allow for targeted blood flow to the ipsilateral limb during surgical and/or cardiac procedures while still providing ample blood flow to the head and body. Stated differently, a balloon may be configured to occlude or partially occlude a blood vessel in which a bidirectional flow cannula resides and this occlusion, or partial occlusion of the vessel may facilitate bidirectional blood flow from the bidirectional flow cannula's partitioned proximal and distal blood flow ports when the balloon is fully or partially inflated. This may address and/or mitigate clinical risks of ischemic limb conditions seen in patients undergoing extracorporeal circulation using femoral cannulation while not introducing new risks.

Disclosed herein are bidirectional flow cannulas that are configured for insertion into a vessel of a body (e.g., an artery (e.g., femoral artery) and/or vein), oftentimes positioned in a limb, while for example, providing cardiopulmonary support and/or extracorporeal circulation (e.g., cardiopulmonary bypass or extracorporeal membrane oxygenation (ECMO)) to a patient. The bidirectional flow cannulas are configured to deliver the bodily fluid (e.g., blood) to the body as well as to the canulated limb. Supplying the canulated limb with blood via one of the bidirectional flow cannulas disclosed herein may reduce, or otherwise mitigate risks and complications caused by ischemic conditions in the canulated limb and/or reperfusion injuries for the patient following decannulation. Flow of blood to the canulated limb is facilitated by use of a partially, or fully, occluding device positioned on the canula proximate to one or more ports positioned within a main cannula tube from which the blood may exit the cannula and be delivered to the limb. The full, or partial, occlusion by the occluding device may prevent blood exiting from the port from being swept up into the blood stream and delivered away from the limb. This provision of blood to the limb may prevent ischemia of the limb. On some occasions, the occluding device may be configured to disrupt the fluid dynamics of fluid traveling and/or being pushed through the vessel by, for example, creating turbulence and/or back pressure. The ports may be positioned within a sidewall of the main cannula tube proximal to the cannulated limb.

Various devices may be used as the occluding device such as (but not limited to) balloons and skirts that surround the main canula tube. In some embodiments, the balloons may be inflated with, for example, air, gas, and/or liquid (e.g., saline). Additionally, or alternatively, the balloons may be self-inflating and/or may expand when placed within the vessel via, for example, a memory of the material comprising the balloon or filling the balloon when fluid flows through the main lumen. The skirts may be configured to transition from a closed state (prior to seating within the vessel) to an expanded, or flared, state when in the vessel thereby partially, or wholly, occluding the vessel.

Many of the bidirectional flow cannulas described herein may include a supporting, or anti-kinking, element such as a wire or coil embedded within and spiraling around a main cannula tube. The supporting element may be made from, for example, stainless steel, fluorinated ethylene propylene (FEP), or other similar rigid, but flexible, material. On some occasions, the supporting material may have a diameter that is slightly (e.g., 0.05-0.7 mm) smaller than an inner diameter of the main cannula tube so that the supporting material extends down into a central, or main, lumen of the main cannula tube, which may alter the flow of blood through the main cannula tube by for example, introducing turbulence into the blood flow. This turbulence may, in some instances, increase a rate of blood flow through the ports and/or reduce hemolysis for blood flowing through the main lumen.

In some instances, the supporting element may be positioned under, or over, a suction line. Additionally, or alternatively, the supporting element may be configured to provide varying stiffness along the length of the bidirectional flow cannula by, for example, varying a pitch of the supporting element, varying a thickness and/or material used for the supporting element along the length of the bidirectional flow cannula, and/or using a wire that is ribbon or laser cut.

In some embodiments, the bidirectional flow cannulas disclosed herein may include a marker that may be visible to an imaging technology such as a radio-opaque marker. The marker may assist with identification of a position of the marker (and therefore the bidirectional flow cannula) within the body of the patient.

In many cases the bidirectional flow cannulas disclosed herein will be mechanical coupled to a machine supplying blood to the bidirectional flow cannula, such as a heart and lung machine. This mechanical coupling may be achieved by one or more connecters that, in some instances, may be configured with one or more de-airing ports, valves, and/or lines configured to, for example, allow air in the bidirectional flow cannula to escape.

At times, the bidirectional flow cannulas disclosed herein may be configured to, for example, reduce thrombosis, improve tactile response, improve kink resistance, and/or increase ease of insertion and/or delivery of the bidirectional flow cannula. The bidirectional flow cannulas disclosed herein may be made from, for example, polyurethane, elast-eon, silicon, pebax, and combinations thereof. In some instances, the bidirectional flow cannulas disclosed herein may be coated with, for example, a friction-reducing coating such as TEFLON or PTFE.

In some embodiments, the bidirectional flow cannulas disclosed herein may include and/or cooperate with a mechanism by which to monitor blood flow to, and/or blood pressure within, a cannulated limb so that, for example, an operator may know how much blood is reaching the cannulated limb so that, for example, risks of ischemia may be recognized and timely mitigated. Exemplary blood flow mechanisms include, but are not limited to, flow meters posited within a bidirectional flow cannula, a device positioned on the limb that monitors blood flow, and/or an oximetry device.

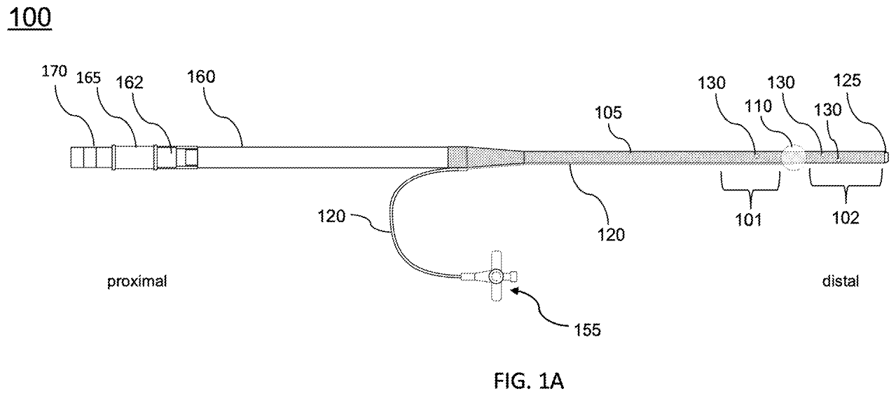

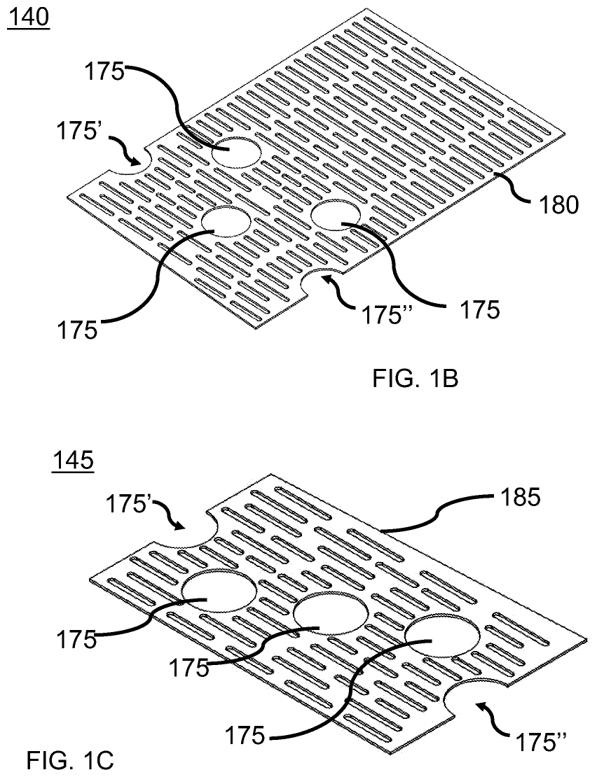

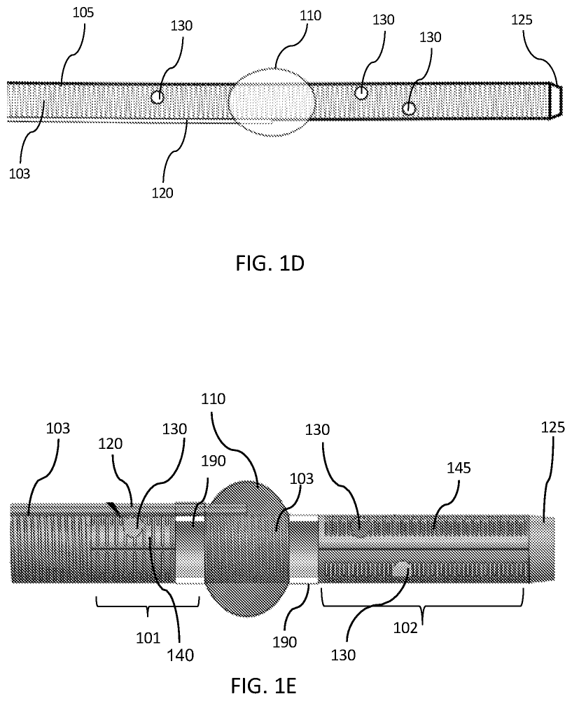

Turning now to the figures, FIG. 1A is a side view of an exemplary bidirectional flow cannula 100 having a proximal and distal side as shown. Bidirectional flow cannula 100 includes a first reinforced region 101, a second reinforced region 102, a support ribbon 103 (shown in FIGS. 1D and 1E), a main cannula tube 105, a balloon 110, an inflation line 120, a tip 125, a plurality of ports 130, a proximal basket 140 (shown in FIGS. 1B and 1E), a distal basket 145 (shown in FIGS. 1C and 1E), an inflation line coupling 155, a tube 160, an optional joint 165 with a plug 162, and an optional coupling 170. Exemplary dimensions for bidirectional flow cannula 100 include an overall length of 380-430 mm, a length of main cannula tube 105 of are an exterior diameter of main cannula tube are 105 4.5-8 mm, exemplary dimensions for a length of main cannula tube 105 are and exemplary dimensions for an exterior diameter of inflation line 120 are 0.7-2 mm. Bidirectional flow cannula 100 may be used to, for example, supply blood to a patient's artery or vein during, for example, ECMO.

Main cannula tube 105 has a central lumen defined by a sidewall of main cannula tube 105. Main cannula tube 105, or a portion thereof, may be configured to reside within a vessel (e.g., artery and/or vein) of a patient during use and provide blood or other fluids to the vessel via, for example, liquid communication with a source of blood/fluid, such as a heart/lung machine, and/or while ECMO is being performed, wherein the blood and/or fluid flows through the central lumen of main cannula tube 105. Main cannula tube 105 may include first reinforced region 101, ribbon 103, balloon 110, second reinforced region 102, and tip 125. Ribbon 103 may be configured to provide stiffness to main cannula tube 105 to, for example, prevent compression, kinking, twisting, and/or bending of main cannula tube 105 while maintaining enough flexibility of main cannula tube 105 to enable desired bending during, for example, an insertion process and/or while resident within a patient's vessel.

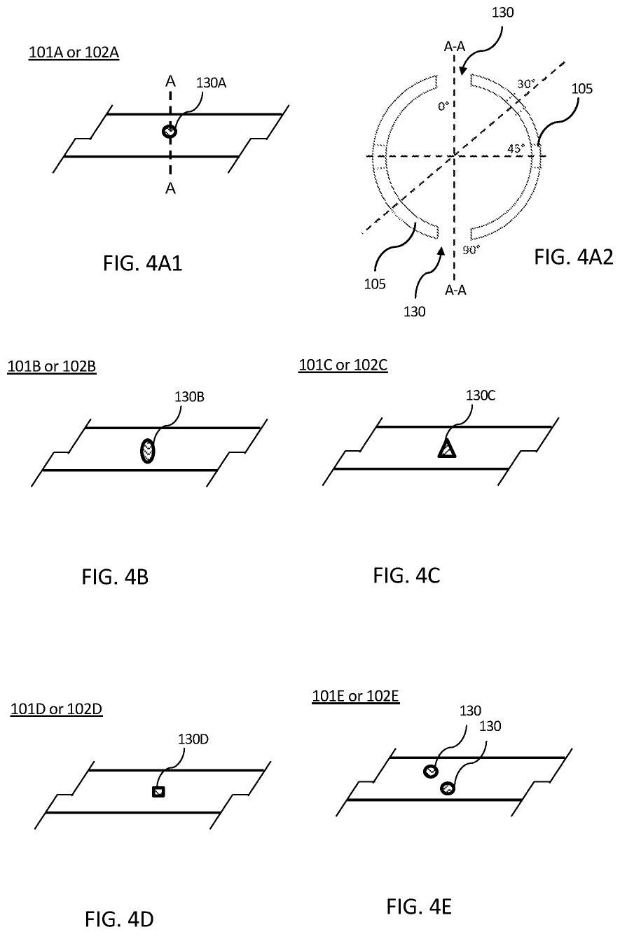

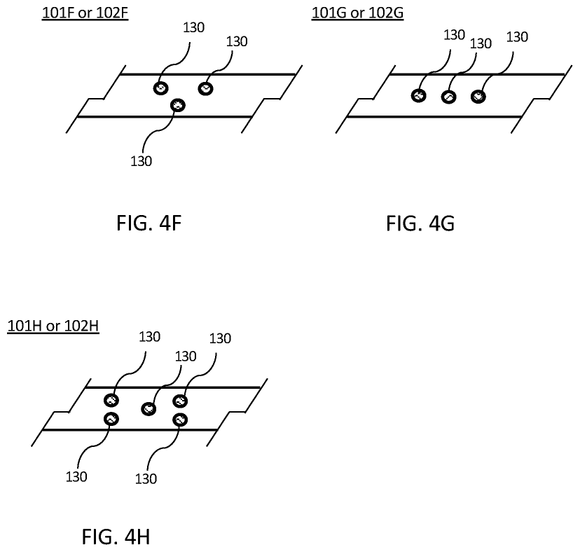

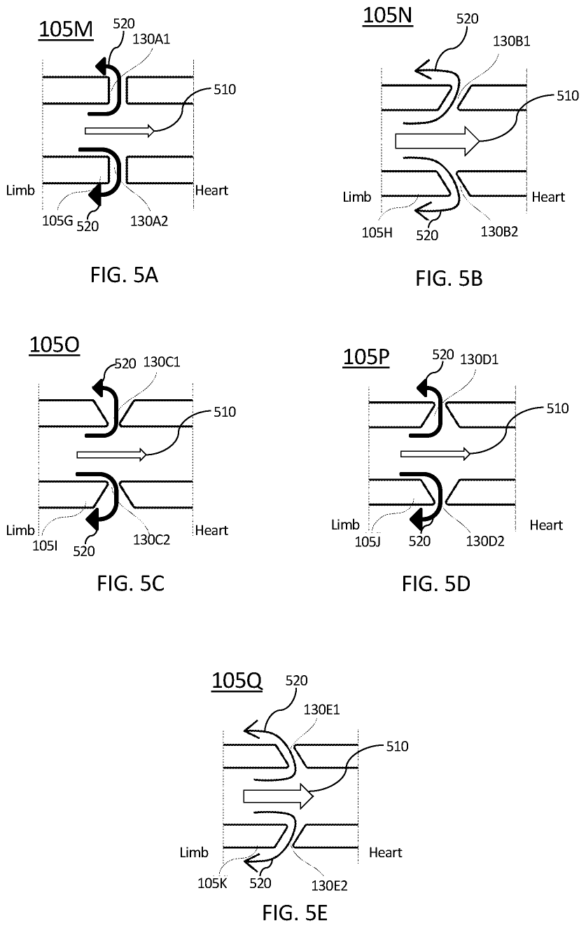

Proximal basket 140 may reside within first reinforced region 101 and may, for example, be imbedded within a sidewall of first reinforced region 101 and/or may be positioned to reside along an interior sidewall of first reinforced region 101 as shown in FIG. 1D. First reinforced region 101 may include a plurality of ports 130 positioned on a proximal side of bidirectional flow cannula 100. Ports 130 that may be openings and/or holes within first reinforced region 101 and proximal basket 140 configured to allow a flow of fluid/blood therethrough as, for example, discussed below. Additional details regarding proximal basket 140 are shown in FIG. 1B and discussed below. Additional details regarding different features and/or configurations of ports 130 are shown in FIGS. 4A-5F.

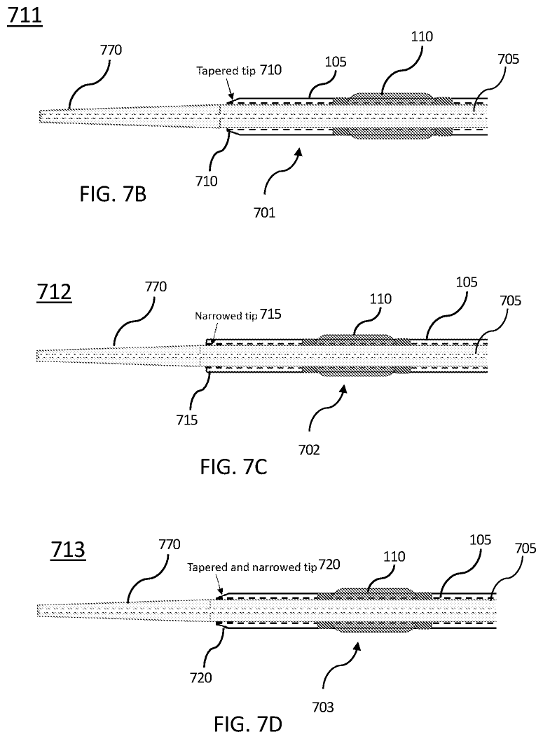

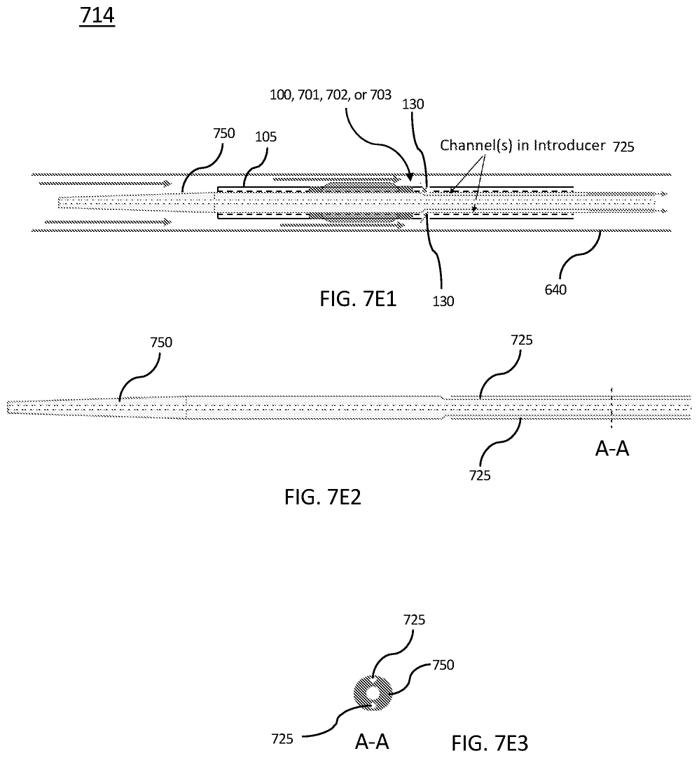

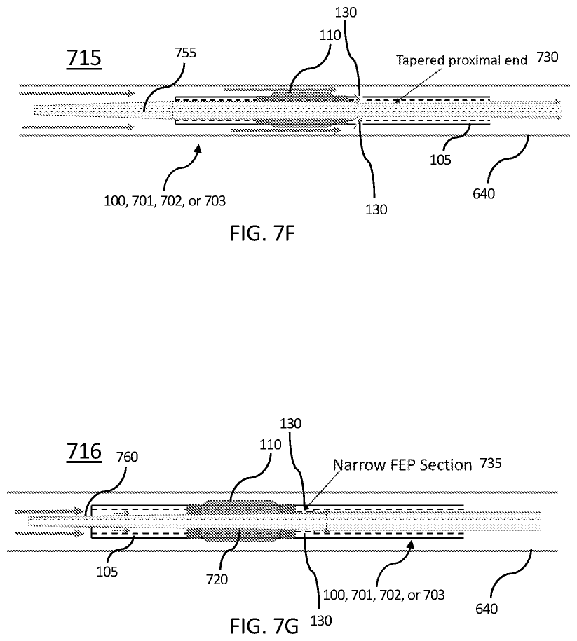

A distal basket 145 may reside within second reinforced region 102 and may, for example, be imbedded within a sidewall of second reinforced region 102 and/or may be positioned to reside along an interior sidewall of second reinforced region. Additional details regarding distal basket 145 are shown in FIG. 1C and discussed below. Second reinforced region 102 may include a plurality of ports 130 that may be openings and/or holes within second reinforced region 102 and distal basket 145. Ports 130 may be configured to allow a flow of fluid/blood therethrough as, for example, discussed below. Second reinforced region 102 terminates with tip 125. Tip 125 may be tapered to, for example, facilitate insertion into the patient's vessel and, in some embodiments, may be flexible and/or elastic. Additional details regarding exemplary configurations of tip 125 are provided in FIGS. 7B-7G and discussed below.

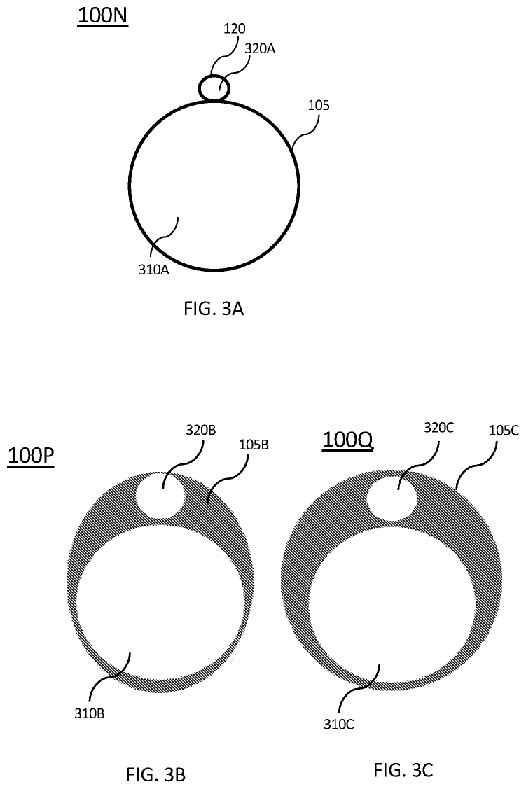

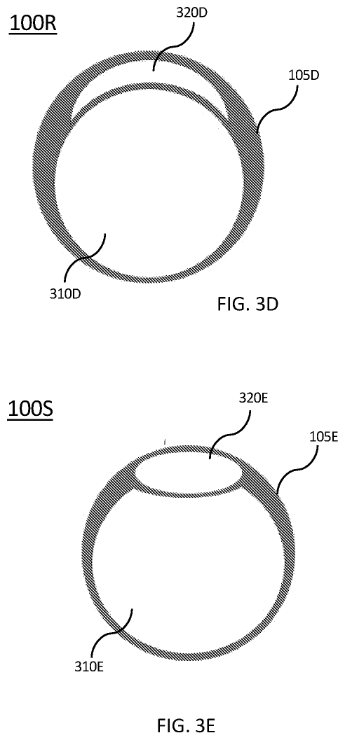

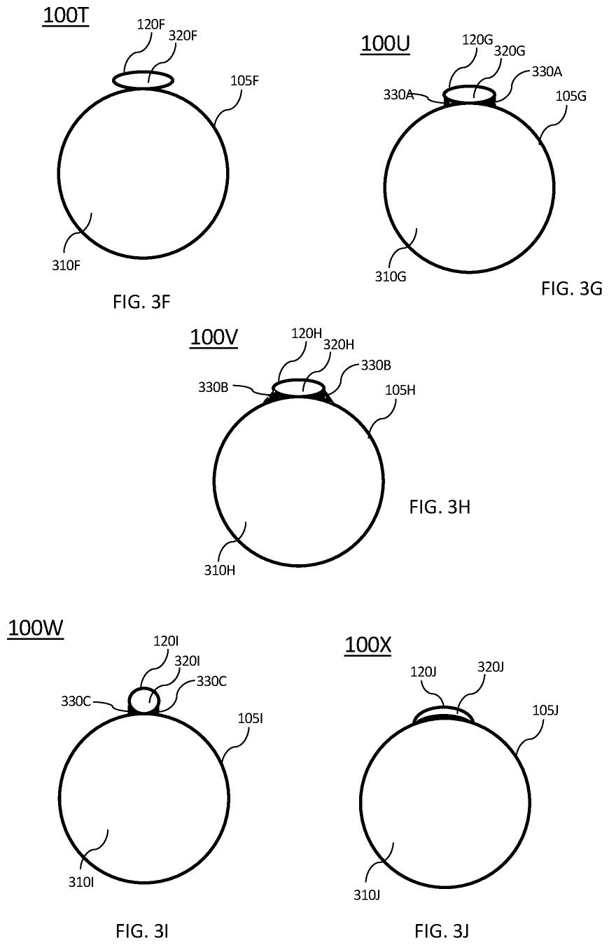

Inflation line coupling 155 is configured to couple to an inflation/deflation mechanism such as an air and/or vacuum pump (not shown) that may push air and/or liquid into inflation line 120 for communication to, and inflation of, balloon 110 once bidirectional flow cannula 100 is placed in situ within a patient's blood vessel and/or while bidirectional flow cannula 100 resides within the patient's blood vessel. Inflation line 120 may also be configured to facilitate deflation of balloon 110 via, for example, evacuation of inflation gas and/or liquid from balloon 110 when, for example, negative pressure (e.g., vacuum) is applied to inflation line 120 by, for example, a vacuum pump. Exemplary inflation line couplings 155 include, but are not limited to, push-in couplings, luer locks, and screw-on couplings. Prior to extraction of bidirectional flow cannula 100 from the patient's vessel (or on other occasions), balloon 110 may be deflated by an application of negative pressure (or vacuum) by the inflation/deflation mechanism to inflation line 120 so that gas and/or liquid may be evacuated from balloon 110 thereby deflating balloon 110. As may be seen in FIG. 1B, inflation line 120 may reside on, and may be coupled to, an exterior surface of main cannula tube 105. Other exemplary configurations or embodiments of inflation line 120 are provided by FIGS. 3A-3J and discussed below.

Tube 160 may be configured to couple to a tapered portion of main cannula tube 105 and/or may extend from a tapered portion of main cannula tube 105. Tube 160 and/or the tapered region of main cannula tube 105 may be configured to reside outside the body when bidirectional flow cannula 100 is being used to provide blood or other fluids to a patient or is otherwise inserted into a vessel.

Optional joint 165 may be sized, shaped, and configured to facilitate easy grasping of bidirectional flow cannula 100 so that optional coupling 170 may be manually inserted into and/or extracted from a tube (not shown) that may be supplying blood or other fluids to bidirectional flow cannula 100. In some embodiments, optional joint 165 may be inserted into tube 160 and/or retained into tube 160 via plug 162 which may be a friction-based coupling with a central lumen therethrough that aligns with a central lumen of main cannula tube 105 (not shown). Approximate dimensions for coupling 170 may be, for example, 0.2-0.8 inches and, in some embodiments, may be a ⅜ inch connector.

FIG. 1B provides a top-perspective view of proximal basket 140 as a flat sheet prior to being rolled and deployed within first region 101 of main cannula tube 105 as shown in FIGS. 1A and 1B. Proximal basket 140 includes a plurality of rounded rectangular perforations, three holes 175 arranged in a triangular formation, a first partial hole 175′ and a second partial hole 175″ as shown. When proximal basket 140 is rolled into a cylinder prior to deployment in first region 101, first partial hole 175′ and second partial hole 175″ may meet and form a complete hole like holes 175. Proximal basket 140 may be, for example, a sheet of stainless steel, or other appropriate material, and the perforations and/or holes 175 therein may be produced via, for example, stamping and/or laser cutting. The rounded rectangular perforations of proximal basket 140 may be configured to provide flexibility to proximal basket 140 when deployed within first region 101. Holes 175 as well as first and second partial holes 175′ may be sized, positioned, and configured to align with the holes or openings in first region 101 that form ports 130 so that proximal basket 140 does not occlude any of the ports 130 of first region 101.

FIG. 1C provides a top-perspective view of distal basket 145 as a flat sheet prior to being rolled and deployed within second region 102 of main cannula tube 105. Distal basket 145 includes a plurality of rounded rectangular perforations, three holes 175 arranged in a diagonal and linear fashion, a first partial hole 175′ and a second partial hole 175″ as shown. When distal basket 145 is rolled into a cylinder prior to deployment in second region 102, first partial hole 175′ and second partial hole 175″ may meet and form a complete hole like holes 175. Distal basket 145 may be, for example, a sheet of stainless steel, or other appropriate material, and the perforations and/or holes 175 therein may be produced via, for example, stamping and/or laser cutting. The rounded rectangular perforations of distal basket 145 may be configured to provide flexibility to distal basket 145 when deployed within second region 102. Holes 175 and first and second partial holes may be sized, positioned, and configured to align with the holes or openings in second region 102 of ports 130 so that distal basket 145 does not occlude any of the ports 130 of second region 102.

Proximal basket 140 and/or distal basket 145 may be configured and arranged to provide additional support to and/or scaffolding for first region 101 and second region 102, respectively, that may operate to, for example, prevent kinking, bending, or twisting of first region 101 and/or second region 102, respectively. This may be of particular importance within first and second regions 101 and 102 because their respective structural integrity may be compromised by ports 130 and proximal and/or distal baskets 140 and/or 145 may compensate for the decreased structural integrity of main cannula tube caused by ports 130. Proximal basket 140 and/or distal basket 145 may be embedded within a sidewall of first region 101 and second region 102, respectively, and, in some cases, may be overlaid upon, or reside underneath, ribbon 103 when, for example, ribbon 103 runs along a length of first region 101 and second region 102, respectively.

FIG. 1D provides a side view of a portion of main cannula tube 105 without proximal or distal baskets 140/145 so that a manner in which ribbon 103 extends in a spiral-like fashion along a length of main cannula tube 105 may be seen. FIG. 1E is a close-up view of a distal portion of bidirectional flow cannula 100 that includes first region 101 and second region may be seen. As may be seen in FIG. 1E, proximal basket 140 extends through first region 101 and may reside within a side wall of first region 101 either on top of, or below, ribbon 103. Distal basket 145 may extend through second region 102 and may reside within a side wall of second region 102 either on top of, or below, ribbon 103. In some embodiments, ribbon 103 may not extend through first and/or second region 101 and/or 102. FIG. 1E also shows optional reinforced regions 190 on either side of balloon 110. Optional reinforced regions 190 may operate to affix balloon 110 to an exterior diameter of main cannula tube 105 and/or further prevent kinking or bending of bidirectional flow cannula 100 at, or near, balloon 110.

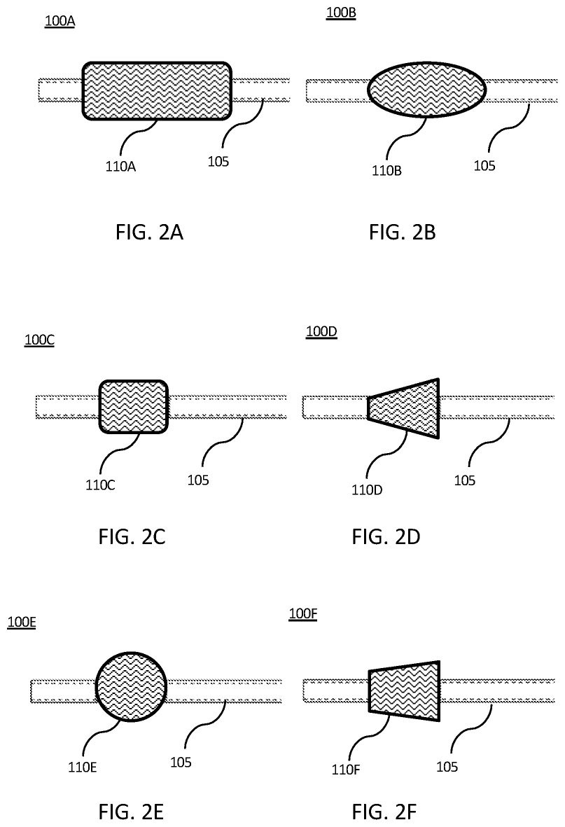

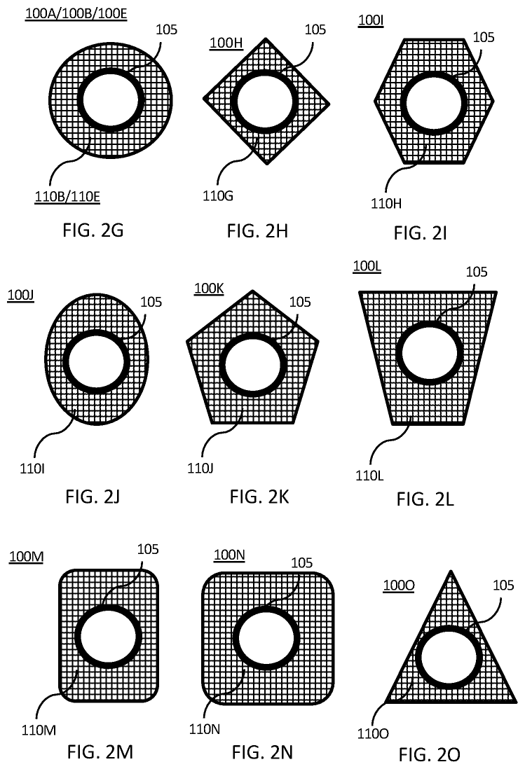

FIGS. 2A-2F are side views of a portion of six exemplary bidirectional flow cannulas 100, that include first-sixth balloons 110A-110F of different sizes and shapes that circumferentially surround a main canula tube 105 with an exterior wall of main cannula tube 105 being illustrated with a solid line and an interior wall of main cannula tube 105 being illustrated with a dashed, or broken, line. First-sixth balloons 110A-110F may be made from any flexible material (e.g., silicon, plastic, etc.) capable of inflating and holding air, gas, and/or liquid (e.g., saline or contrast) and deflating or releasing the air/gas/liquid held within the balloon via, for example, an inflation line like inflation line 120 (not shown). Further details regarding exemplary balloon inflation mechanisms are provided below with regard to, for example, FIGS. 3A-3J.

In particular, FIG. 2A provides a side view of an exemplary first bidirectional flow cannula 100A that includes main cannula tube 105 and an elongated and circularly shaped balloon 110A; FIG. 2B provides a side view of an exemplary second bidirectional flow cannula 100B that includes main cannula tube 105 and an oval-shaped balloon 110B; FIG. 2C provides a side view of an exemplary third bidirectional flow cannula 100C that includes main cannula tube 105 and a rounded square-shaped balloon 110C; FIG. 2D provides a side view of an exemplary fourth bidirectional flow cannula 100D that includes main cannula tube 105 and a triangularly shaped balloon 110D; FIG. 2E provides a side view of an exemplary fifth bidirectional flow cannula 100E that includes main cannula tube 105 and spherically shaped balloon 110E; and FIG. 2F provides a side view of an exemplary sixth bidirectional flow cannula 100F that includes main cannula tube 105 and a rhomboid-shaped balloon 110F.

FIGS. 2G-2O provide cross sections of bidirectional flow cannulas 100 with different various balloons 100. The cross-sections are taken in a direction perpendicular to main cannula tube 105 through an approximate center of balloon 100. In particular, FIG. 2G provides a cross section of bidirectional flow cannulas 100A, 100B, and/or 100E that shows a substantially round, or circular, cross section of balloons 110B, and 110E that surround main cannula tube 105. FIG. 2H shows a cross-section of a bidirectional flow cannula 100H with a substantially square-shaped balloon 100H that surrounds main cannula tube 105. FIG. 2I shows a cross-section of a bidirectional flow cannula 100I with a substantially hexagonal balloon 100I that surrounds main cannula tube 105. FIG. 2J shows a cross-section of a bidirectional flow cannula 100J with a substantially ovoid balloon 100J that surrounds main cannula tube 105. FIG. 2K shows a cross-section of a bidirectional flow cannula 100HK with a substantially pentagonal balloon 100K that surrounds main cannula tube 105. FIG. 2L shows a cross-section of a bidirectional flow cannula 100L with a substantially rhomboid balloon 100L that surrounds main cannula tube 105. FIG. 2M shows a cross-section of a bidirectional flow cannula 100M with a substantially rounded-rectangular balloon 100M that surrounds main cannula tube 105. FIG. 2N shows a cross-section of a bidirectional flow cannula 100N with a substantially rounded square balloon 100N that surrounds main cannula tube 105. FIG. 2O shows a cross-section of a bidirectional flow cannula 100O with a substantially triangular balloon 100O that surrounds main cannula tube 105.

Each of the differently shaped balloons 100 shown in FIGS. 2A-2O may operate differently when positioned within a patient's blood vessel. For example, spherical or ovoid shapes of balloons 110B and 110E as in FIGS. 2B and 2E, respectively, may be configured to partially and/or fully occlude the vessel when inflated and positioned within the vessel. Alternatively, a shape of balloon 110A, 110C, 110D, 110F, 110G, 110H, 110J, 110L, 110M, 110N, and/or 100O may fully or partially occlude the vessel. When the vessel is partially occluded, some bodily fluid (e.g., blood) flows past the balloon to, for example, a limb downstream of the canula, which in the case of positioning canula 100 within a blood vessel may assist with facilitating enough blood flow to a limb in which the vessel is present (e.g., a leg or arm) to prevent ischemia of the limb. In some embodiments, a shape and/or size of a balloon 110 may be configured to, for example, disrupt the Bernoulli effect within the vessel (i.e., as velocity increases from the main lumen of the cannula, the pressure decreases). This may cause the blood in the limb to be pulled from a region of relatively high blood pressure to region of relatively low blood pressure, towards the head and body, which may allow a dedicated source of fluid, or blood, to the limb to be established.

In some embodiments, one or more of the balloons shown in FIGS. 2A-2O (e.g., balloon 110B, 110D, 110F, 110G, 110H, 110J, 110L, 110M, and/or 110O) may be configured to provide less apposition to a vessel wall in which a corresponding bidirectional flow cannula is resident when fully or partially occluded. This may act to, for example, decrease a likelihood of the bidirectional flow cannula and/or a balloon thereof causing, or contributing to, development of intimal hyperplasia and/or damage to the vessel wall.

Additionally, or alternatively, one or more of the balloons (e.g., balloon 110A, 110B, 110C, 110M, and/or 110N), shown in FIGS. 2A-2O may be configured to distribute any force encountered by a vessel in which a corresponding bidirectional flow cannula is resident over a relatively (compared with, for example, a spherically shaped balloon) larger surface area, which may decrease a likelihood of vessel damage.

Additionally, or alternatively, one or more of the balloons shown in FIGS. 2A-2O may be configured to reduce a likelihood of occluding branching blood vessels proximate to a location of the bidirectional flow cannula. This may be accomplished by, for example, having a balloon with a relatively small surface area (e.g., balloon 110E or 110C) and/or a balloon that may not fully occlude the vessel (e.g., balloon 110D, 110F, 110G, 110H, 110J, 110L, 110M, and/or 110O). Additionally, or alternatively, one or more of the balloons (e.g., balloon 110B, 110D, 110F, 110H, 110K, 110L, 110M, and/or 110O) shown in FIGS. 2A-2O may be configured to improve blood flow mechanics around and/or past the balloon, which may, for example, decrease a likelihood of a thromboembolic event.

Additionally, or alternatively, one or more of the balloons (e.g., balloon 110A, 110C, 110M and/or 110N) shown in FIGS. 2A-2O may be configured to provide support to a vessel wall in which a corresponding bidirectional flow cannula is resident. This support may assist with prevention of a blockage of one or more of ports 130 in the event of, for example, vessel spasm and/or collapse. Additionally, or alternatively, one or more of the balloons (e.g., balloon 110A, 110B, 110C, 110M, and/or 110N), shown in FIGS. 2A-2O may be configured to support to the bidirectional flow cannula and/or hold it in place so that it remains in place during use.

In some embodiments, one or more of the balloons shown in FIGS. 2A-2O (e.g., balloon 110B, 110D, 110F, 110G, 110H, 110J, 110L, 110M, and/or 110O) may be configured to have less surface area of the balloon in contact with a vessel wall in which a corresponding bidirectional flow cannula is resident when fully or partially occluded. This may act to, for example, decrease a likelihood of the bidirectional flow cannula and/or a balloon thereof causing, or contributing to, development of intimal hyperplasia, stress to the vessel wall, and/or damage to the vessel wall.

FIGS. 3A-3J each provide a cross-section exemplary bidirectional flow cannulas 100 and 100P-100V, respectively, taken at a vertical cross section of main cannula tube 105 and inflation lumen 120 that is perpendicular to main cannula tube 105 and inflation lumen 120. The cross-section may be taken at any point along a length of main cannula tube 105 between the tapered region and the balloon 110. Each of FIGS. 3A-3J shows a sidewall of main cannula tube 105 and a main lumen 310 positioned inside the sidewall defining main cannula tube 105. The main lumens 310 may be configured to allow for the flow of fluids (e.g., blood) therethrough. Each of FIGS. 3A-3J also show different configurations for inflation line 120 that include an inflation line lumen 320 configured to facilitate a flow of air, gas, and/or liquid into and/or out of balloon 110 (not shown) to facilitate inflation and deflation of balloon 110. inflation line lumens 320 may be configured with interior diameters ranging from, for example, 0.5-5 mm and exterior diameters ranging from, for example, 0.7-7 mm.

In particular, FIG. 3A provides a cross-section view of bidirectional flow cannula 100 as shown in FIG. 1A and shows the main cannula tube 105 of FIG. 1A and also illustrates a first lumen 310A enclosed within the sidewall of main cannula tube 105A. FIG. 3A also shows inflation line 120 (as shown in FIG. 1A) positioned on top of (as oriented in FIG. 3A) main cannula tube 105 and affixed thereto via, for example, heat, chemical, and/or vibration bonding. Additionally, or alternatively, bidirectional flow cannula 100 may be manufactured so that main cannula tube 105 and inflation line 120 are simultaneously extruded and/or molded (e.g., injection molding) by manufacturing equipment.

In some instances, the configuration shown in FIG. 3A may cause problems when used in a vessel such as leaking and/or movement (e.g., oscillations left and right (as oriented in FIG. 3A)) of first inflation line 120A that may cause abrasions on the inside of the vessel and/or provide a location where the fluid may collect and/or clot. These problems may be mitigated and/or nearly eliminated by the bidirectional flow cannula 100P-100V configurations of FIGS. 3B-3J, respectively. For example, FIG. 3B shows another exemplary bidirectional flow cannula 100P that includes a second inflation line lumen 320B that is resident within a second exemplary main cannula tube 105B that surrounds both second inflation line lumen 320B and a second main lumen 310B. In another example, FIG. 3C shows a cross-section view of an exemplary bidirectional flow cannula 100Q that is similar to bidirectional flow cannula 100P except that a relative thickness of third main cannula tube 105C is larger (i.e., thicker) than the thickness of second main cannula tube 105B. Inflation line lumen 320B and 320C have an approximately circular cross-section.

FIGS. 3D-3J provide cross-section views of exemplary inflation lines that have a non-circular cross section. In particular, FIG. 3D shows a cross-section view of an exemplary bidirectional flow cannula 100R that includes a fourth main cannula tube 105D, which has a substantially circular outer circumference and a crescent-shaped shaped inflation line lumen 320D positioned with a sidewall of bidirectional flow cannula 100R proximate to a fourth main lumen 310D as shown. FIG. 3E shows a cross-section view of an exemplary bidirectional flow cannula 100S that includes a fifth main cannula tube 105E, which has a substantially circular outer circumference with an oval-shaped shaped inflation line lumen 320E positioned within a sidewall of bidirectional flow cannula 100S proximate to a fifth main lumen 310E as shown. FIG. 3F shows a cross-section view of an exemplary bidirectional flow cannula 100T that includes a sixth main cannula tube 105F with sixth main lumen 310F positioned in an approximate center of sixth main cannula tube 105F and an oval-shaped shaped inflation line 120F with a correspondingly oval-shaped inflation line lumen 320F positioned therein. Sixth inflation line 120F is positioned on an exterior surface of sixth main cannula tube 105F as shown and may be affixed thereto in a manner similar to how inflation line 120 is affixed to main cannula tube 105 as shown and described above with regard to, for example, FIG. 3A. FIG. 3G shows a cross-section view of an exemplary bidirectional flow cannula 100U that includes a seventh main cannula tube 105G with a seventh main lumen 310G positioned in an approximate center of seventh main cannula tube 105G, an oval-shaped shaped inflation line 120G with a correspondingly oval-shaped inflation line lumen 320G positioned therein. The embodiment of FIG. 3G also includes two inflation line lumen supports 330A positioned on either side (left and right as shown in FIG. 3G) of inflation line 120G between a lower (as oriented in FIG. 3G) exterior surface of inflation line 120G and an upper (as oriented in FIG. 3G) exterior surface of main cannula tube 105G. Supports 330A may be configured to support inflation line 120G so that it, for example, does not move (e.g., left, or right) independently of main cannula tube 105G, provide a location for the collection of clotted blood, and/or cause irritation when bidirectional flow cannula 100U is in a patient's vessel. In some embodiments, supports 330, such as support 330B shown in FIG. 3H may be configured to smooth a vertical cross-sectional profile of bidirectional flow cannula 100V so that an outer profile of an inflation line 120H blends into an outer profile of a main cannula tube 105H as shown in FIG. 3H. FIG. 3H also shows a main lumen 310H positioned within main cannula tube 105H and an inflation line lumen 320H positioned within inflation line 120H. FIG. 3I is a cross section of a bidirectional flow cannula 100W that includes a circular inflation line 120I with a corresponding inflation line lumen 320I positioned therein. Inflation line 120I is positioned on an exterior surface of a main cannula tube 105I with a main lumen 310I and is supported on either side by supports 330C, which are similar to supports 330A and 330B with the exception that their shape is adjusted to accommodate the different shape of inflation line 120I. FIG. 3J provides a vertical cross-section view of an exemplary bidirectional flow cannula 100X that includes a crescent shaped inflation line 120J that includes a corresponding crescent shaped inflation line lumen 320J positioned therein. Inflation line 120J extends from an exterior surface of a main cannula tube 105X with a main lumen 310J.

On some occasions, a position and/or orientation of an inflation line 120 and/or inflation line lumen 320 may serve as a marker visible to a clinician that helps them see the inflation line and/or inflation line lumen and/or identify an orientation of a bidirectional flow cannula, and corresponding ports (e.g., ports 130), during insertion and/or use the bidirectional flow cannula. For example, bidirectional flow cannulas 100R, 100S, 100T, 100U, 100V, and/or 100X have inflation lines/lumens that are relatively large, which may assist a clinician in seeing and/or visually identifying the inflation line and/or inflation line lumen.

In some embodiments (e.g., bidirectional flow cannula 100P, 100Q, 100R, 100S, 100J, 100V, 100W, and/or 100X), a size, shape, and/or configuration of an inflation line 120 and/or inflation line lumen 320 may decrease an overall outer diameter/profile of a bidirectional flow cannula and/or main cannula tube. This may, for example, limit irritation caused by an inflation line that may extend from a main cannula tube and press into and/or rub against a vessel wall and/or decrease a likelihood of blood leaking out in a channel positioned at a junction between an inflation lumen and the main cannula tube (see e.g., bidirectional flow cannula 100A and/or 100T) when these bidirectional flow cannulas are implanted in the blood vessel.

Additionally, or alternatively, in some embodiments (e.g., bidirectional flow cannula 100R, 100S, and/or 100X) the crescent shaped inflation line/lumen may make a corresponding main cannula tube more flexible, where the rounder inflation line/lumen may make a corresponding main cannula tube less flexible, which may be advantageous in situations where the inflation line/lumen acts like a spine for the main cannula tube. Additionally, or alternatively, in some embodiments (e.g., bidirectional flow cannula 100P, 100Q, 100R, 100S, and/or 100X), a shape of the inflation line and/or inflation line lumen may require less adhesive, be easier to manufacture, and/or provide more flexibility to the main cannula tube.

FIGS. 4A1-4H provide illustrations for various port shapes and arrangements within main cannula tube 105 and, in particular, within first region 101 and or second region 102 of main cannula tube 105. Ports 130 may be made via any appropriate means including, but not limited to, punching, scoring, cutting, and/or molding. Ports 130 may be of any appropriate size and/or shape (e.g., circular, oval, triangle, or square) or combination thereof. In some embodiments, one or more ports 130 on a proximal side of balloon 110 may be smaller than one or more ports 130 on a distal side of balloon 110 and vice versa. In some embodiments, a quantity of ports (e.g., 2-16) on a proximal side of balloon 110 may be smaller than a quantity of ports 130 on a distal side of balloon 110 (e.g., 1-15). Alternatively, a quantity of ports (e.g., 2-16) on a distal side of balloon 110 may be smaller than a quantity of ports 130 on a proximal side of balloon 110 (e.g., 1-15). A size and/or shape of hole 175 of proximal and distal basket 140 and 145 may be sized, shaped, and arranged to correspond to holes or openings in main cannula tube 105 that correspond to ports 130.

For example, ports 130 may be shaped, sized, and/or configured to, for example, direct exiting blood flow in a particular direction, reduce a likelihood of clotting, and/or control a speed and/or velocity of blood flowing out of a port 130 and/or tip 125. Ports 130 may be arranged any orientation/angle; some of which are shown in FIG. 4A2 and FIGS. 5A-5F. FIG. 4A1 provides a top view of a portion of a first region 101 and/or second region 102 of bidirectional flow cannula 100 that includes a port 130A with an approximately circular shape and FIG. 4A2 is a cross-section view of bidirectional flow cannula 401 taken along bisecting line A-A. On some occasions, ports 130 may be positioned circumferentially around a main cannula tube 105 and/or on a top and a bottom of a main cannula tube 105 as shown in FIG. 4A2, which shows a first port 130 positioned on a top of main cannula tube 105 (as oriented in FIG. 4A2) and a second port 130 positioned on a bottom of main cannula tube 105 (as oriented in FIG. 4A2).

FIGS. 4B-4D provide top views of exemplary first and/or second region 101B, 101C, 101D, 102B, 102C, and 102D, respectively, that include ports 130 of different shapes, wherein FIG. 4B is a top view of first region 101B and/or second region 102B with an oval-shaped port 130B; FIG. 4C is a top view of first region 101C and/or second region 102C with a triangularly-shaped port 130C; and FIG. 4D is a top view of first region 101D and/or second region 102D with a square-shaped port 130D. It is noted that a bidirectional flow cannula may use any one or more of these shapes (or different shapes) for ports 130 and that they may be used in combination (e.g., circular, and triangular; square and oval, etc.) within the same region or in different regions (i.e., first region 101 or second region 102).

FIGS. 4E-4H provide top views of exemplary first and/or second region 101E, 101F, 101G, 101H, 102E, 102F, 102G, and 102H, respectively, that each include an array of ports 130 arranged in different configurations. For ease of illustration, the port shape shown in FIGS. 4E-4H is that of a circular port like circular port 130 of FIG. 4A1 but it is to be understood that a shape of a port shown in the arrays of FIGS. 4E-4H may be of any shape including, but not limited to, the oval, triangular, and square shapes shown in FIGS. 4B-4D. In particular, FIG. 4E is a top view of first region 101E or second region 102E that includes an array of two ports 130 arranged in a diagonal and linear fashion; FIG. 4F is a top view of first region 101F or second region 102F that includes an array of three ports 130 arranged in a triangular fashion; FIG. 4G is a top view of first region 101G or second region 102G that includes an array of three ports 130 arranged in a linear fashion; FIG. 4H is a top view of first region 101H or second region 102H that includes an array of five ports 130 arranged in a “X”-like fashion. In some embodiments, a bidirectional flow cannula may include different arrays (e.g., quantity of ports and/or arrangement of ports within main cannula tube 105) for the first region 101 and the second region 102. For example, a bidirectional flow cannula may include first region 101F and second region 102H. Alternatively, a bidirectional flow cannula may include first region 101D and second region 102G or a bidirectional flow cannula may include first region 101G and second region 102H.

Ports 130 may be cut and/or manufactured into the body of main cannula tube 105 at different angles and/or orientations in order to, for example, direct exiting blood flow in a particular direction, reduce a likelihood of clotting, and/or control a speed and/or velocity of blood flowing out of a port 130. On some occasions, an angle or orientation of edges of a port 130 may be the same and, at other times, a port 130 may have two or more edges that are oriented and/or cut at different angles relative to one another. For example, FIGS. 5A-5E provide cross-section views of a portion of exemplary main cannula tube 105M, 105N, 105O, 105P, and 105Q, respectively, each with a pair of ports 130 disposed therein.

More particularly, FIG. 5A provides a cross-section view of a portion of main cannula tube 105M with a first pair of ports 130A1 and 130A2 that have a right and left side (as oriented in the figure) that are oriented at an angle of approximately 90 degrees relative to the exterior surface of main cannula tube 105M as shown. FIG. 5A also shows a central, or main blood flow 510, that travels through a lumen of main cannula tube 105M toward a patient's heart and also shows two secondary blood flows 520 that exit from the first pair of ports 130B1 and 130B2 and travel toward the patient's limb.

FIG. 5B depicts a cross-section view of a portion of an exemplary main cannula tube 105N with a second pair of ports 130B manufactured at different angles relative to one another wherein a top second port 130B1 has a left and a right (as oriented in the figure) side that are approximately parallel to one another and at an angle of approximately 30-70 degrees relative to the exterior surface of exemplary main cannula tube 105N. Bottom second port 130B2 has a left and a right (as oriented in the figure) side that are oriented approximately parallel to one another and at an angle of approximately 110-160 degrees relative to the exterior surface of main cannula tube 105N. FIG. 5B also shows primary blood flow 510 traveling through a main lumen of main cannula tube 105N and secondary blood flows 520 from each of top second port 130B1 and bottom second port 130B2.

FIG. 5C is a cross-section view of a portion of an exemplary main cannula tube 105O with a third pair of ports 130C manufactured so that a top third port 130C1 has a right side (as oriented in the figure) that is oriented at an angle of approximately 30-70 degrees relative to the exterior surface of exemplary main cannula tube 105O and a left side that is oriented at an angle of approximately 110-160 degrees relative to the exterior surface of exemplary main cannula tube 105O in a point-like configuration so that an inner diameter of top third port 130C1 is smaller than an outer diameter of top third port 130C1 as shown. Bottom third port 130C2 has a right side (as oriented in the figure) that is oriented at an angle of approximately 110-160 degrees relative to the exterior surface of exemplary main cannula tube 105O and a left side that is oriented at an angle of approximately 30-70 degrees relative to the exterior surface of exemplary main cannula tube 105O in a point-like configuration so that an inner diameter of bottom third ports 130C2 is smaller than an outer diameter of bottom third ports 130C2 as shown. FIG. 5C also shows primary blood flow 510 traveling through a main lumen of main cannula tube 105O and secondary blood flows 520 from each of top third port 130C1 and bottom third port 130C2.

FIG. 5D is a cross-section view of a portion of an exemplary main cannula tube 105P with a fourth pair of ports 130D manufactured so that a top fourth port 130D1 has a right side (as oriented in the figure) that is oriented at an angle of approximately 110-160 degrees relative to the exterior surface of exemplary main cannula tube 105P and a left side that is oriented at an angle of approximately 30-70 degrees relative to the exterior surface of exemplary main cannula tube 105P in a point-like configuration so that an inner diameter of top fourth ports 130D1 is larger than an outer diameter of top fourth ports 130D1 as shown. Bottom fourth port 130D2 has a right side (as oriented in the figure) that is oriented at an angle of approximately 30-70 degrees relative to the exterior surface of exemplary main cannula tube 105P and a left side that is oriented at an angle of approximately 110-160 degrees relative to the exterior surface of exemplary main cannula tube 105P in a point-like configuration so that an inner diameter of bottom fourth ports 130D2 is larger than an outer diameter of bottom fourth ports 130D2 as shown. FIG. 5D also shows primary blood flow 510 traveling through a main lumen of main cannula tube 105P and secondary blood flows 520 from each of top fourth port 130D1 and bottom fourth port 130D2.

FIG. 5E is a cross-section view of a portion of an exemplary main cannula tube 105Q with a fifth pair of ports 130E manufactured at different angles to one another wherein a top fifth port 130E1 has a left and a right (as oriented in the figure) side that are manufactured at angles that are approximately parallel to one another and are of approximately 110-160 degrees relative to the exterior surface of exemplary main cannula tube 105Q. Bottom fifth port 130E2 has a left and a right (as oriented in the figure) side that are manufactured at angles that are approximately parallel to one another and are of approximately 30-70 degrees relative to the exterior surface of exemplary main cannula tube 105Q. FIG. 5E also shows primary blood flow 510 traveling through a main lumen of main cannula tube 105Q and secondary blood flows 520 from each of top fifth port 130E1 and bottom fifth port 130E2.

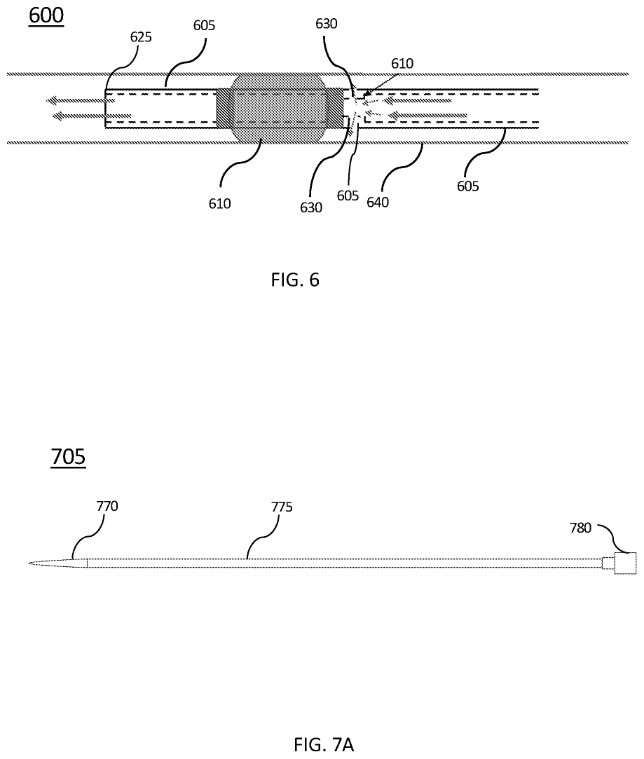

FIG. 6 provides a side view of a bidirectional flow cannula 600 with a main cannula tube 605 that includes a balloon 610, a tip 625, a plurality of ports 630, a narrow segment 610 with a thicker sidewall than a proximate portion of main cannula tube 605 so that main cannula tube 605 has a reduced inner diameter relative to the inner diameter of the remainder of main cannula tube 605 within narrow segment 610. Narrow segment 610 includes two ports 130 positioned on the top and bottom (as oriented in FIG. 6) of narrow segment 610. Narrow segment 610 may be configured to, for example, disrupt laminar blood flow through one or more ports 630 and/or tip 625. Additionally, or alternatively, a configuration of narrow segment 610 may assist with provision of blood flowing to the limb by, for example, causing turbulent flow near ports 630, increasing a velocity of the flow of blood through port 630, and/or decreasing a velocity of blood flowing through tip 625. Balloon 610, tip 625, and plurality of ports 630 may be similar to balloon 110, tip 125, and ports 130 as discussed herein.

On some occasions, the bidirectional flow cannulas disclosed herein may be configured for cooperation with a cannula introducer (also referred to herein as an “introducer”) configured to be inserted through the main lumen of a main cannula tube and assist with insertion of the bidirectional flow cannula into a blood vessel and placement therein. Once the bidirectional flow cannula is in a desired position within the blood vessel via manipulation of the introducer, it is extracted from the bidirectional flow cannula so that blood may flow through the main lumen of the bidirectional flow cannula while it is seated within the blood vessel. FIG. 7A is a side view of an exemplary bidirectional flow cannula introducer 705 that includes a tip 770, a body 775, and a handle 780. Introducer 705 may be configured to assist with the insertion, or introduction, of a bidirectional flow cannula in through a surgical opening in a patient (typically the patient's leg (e.g., femoral artery or vein) or arm) and into a blood vessel of the patient. Introducers 705 may be configured to be flexible but, may be more rigid than the bidirectional flow cannula they are introducing to the vessel so that, for example, the bidirectional flow cannula/introducer system may be maneuvered through tissue and into the target blood vessel without bending, kinking, or twisting. Introducer 705 may provide feedback to the user (tactile, signal with blood flow, etc.) regarding the position of the bidirectional flow cannula in the vessel, such as when the proximal ports are inserted into the vessel.

FIGS. 7B-7G provide cross-section views of exemplary systems 711, 712, 713, 714, 715, and 716, respectively, that include a bidirectional flow cannulas like the bidirectional flow cannulas disclosed herein and introducer 705. In particular, FIG. 7B provides a cross-section view of a first introducer/canula system 711 that includes a bidirectional flow cannula with a tapered tip 701 and an introducer 705. Bidirectional flow cannula with a tapered tip 701 includes balloon 110, a main cannula tube 105 that has a tapered tip, or end, 710 positioned proximate to introducer tip 770. Tapered tip 710 may be configured so that an outer profile, or diameter, of the first introducer/canula system 711 gradually increases in size along its length, which may, for example, make insertion of the first introducer/canula system 711 into a vessel easier, reduce trauma to tissues caused by insertion of the first introducer/canula system 711 into the blood vessel, and/or may prevent the tip of the bidirectional flow cannula 701 from pulling away, or otherwise separating, from introducer 705 upon, for example, insertion of first introducer/canula system 711 into the body (e.g., through skin, muscle, and facia) and/or a blood vessel.

FIG. 7C provides a cross-section view of a second introducer/canula system 712 that includes a bidirectional flow cannula with a narrowed tip 702 and an introducer 705. Bidirectional flow cannula with a narrowed tip 702 includes a balloon 110, main cannula tube 105 that has a narrowed tip, or end, 715 positioned proximate to introducer tip 770. Narrowed tip 715 may be configured so that an outer profile of the first introducer/canula system 712 gradually increases in size along its length, which may, for example, make insertion of the second introducer/canula system 712 easier, reduce trauma to tissues caused by insertion of the second introducer/canula system 712 into the blood vessel and/or surrounding tissue, and/or may prevent narrow tip 715 from pulling away, or otherwise separating, from introducer 705 upon, for example, insertion of second introducer/canula system 711 into the body (e.g., through skin, muscle, and facia) and/or a blood vessel.

FIG. 7D provides a cross-section view of a third introducer/canula system 713 that includes a bidirectional flow cannula with a tapered and narrowed tip 703 and an introducer 705. Bidirectional flow cannula with a tapered and narrowed tip 720 includes balloon 110 and main cannula tube 105 that has a narrowed tip, or end, 715 positioned proximate to introducer tip 770. Tapered and narrowed tip 720 may be configured so that an outer profile of the first introducer/canula system 713 gradually increases in size along its length, which may make insertion of the third introducer/canula system 713 easier, reduce trauma to tissues caused by insertion of the third introducer/canula system 713 into the blood vessel, and/or may prevent the tip of bidirectional flow cannula 703 from pulling away, or otherwise separating, from introducer 705 upon, for example, insertion of third introducer/canula system 711 into the body (e.g., through skin, muscle, and facia) and/or a blood vessel.

FIG. 7E1 provides a cross-section view of a fourth introducer/canula system 714 that includes a bidirectional flow cannula, such as bidirectional flow cannula 100, 701, 702, or 703 and an introducer with a feedback mechanism 750 positioned within blood vessel 640. Introducer with a feedback mechanism 750 may be configured in a manner similar to introducer 705 with the exception that it includes a mechanism that provides feedback to an operator (e.g., surgeon) who is placing bidirectional flow cannula 704 that the fourth introducer/canula system 714 is correctly placed in the blood vessel of the patient. In the embodiment shown in FIG. 7E1, the feedback mechanism is a channel 725 that carries blood that enters ports 130 and travels upward in channel 725 along a length of introducer 750 so that it may be observed as feedback to the operator that the fourth introducer/canula system 714 has been successfully placed within vessel 640. FIG. 7E2 provides a cross-section view of introducer with feedback mechanism 750 that shows the top and bottom channels 725 and FIG. 7E3 is a cross section view taken at line A-A of introducer with feedback mechanism 750 showing the placement of channels 725 within the sidewalls of introducer 750.

FIG. 7F provides a cross-section view of a fifth introducer/canula system 715 that includes a bidirectional flow cannula, such as bidirectional flow cannula 100, 701, 702, or 703 and an introducer with a feedback mechanism in the form of tapered proximal end 755 that is positioned within blood vessel 640. Introducer with the tapered proximal end feedback mechanism 755 may be configured in a manner similar to introducer 705 with the exception that it includes a mechanism that provides feedback to an operator (e.g., surgeon) who is placing the bidirectional flow cannula that the fifth introducer/canula system 715 is correctly placed in the blood vessel of the patient. The feedback mechanism of introducer 755 is provided by a tapered proximal end 730 of introducer that provides a space, or opening, between an inner diameter of bidirectional flow cannula 100, 701, 702, or 703 and tapered proximal end 730. This opening (along the length of fifth introducer/canula system 715) provides a space for blood that enters ports 130 travel up the space that exists between bidirectional flow cannula 704 and introducer 755 along the length of introducer 755 so that it may be observed as feedback to the operator that the fifth introducer/canula system 715 has been successfully placed within vessel 640.

FIG. 7G provides a cross-section view of a sixth introducer/canula system 716 that includes a bidirectional flow cannula, such as bidirectional flow cannula 100, 701, 702, or 703 and an introducer 760 with a narrow FEP, or support section. Narrow FEP section 735 may, in some instances, disturb a flow of blood entering ports 130 and/or flowing through bidirectional flow cannula 704 that may facilitate delivery of blood to a cannulated limb.

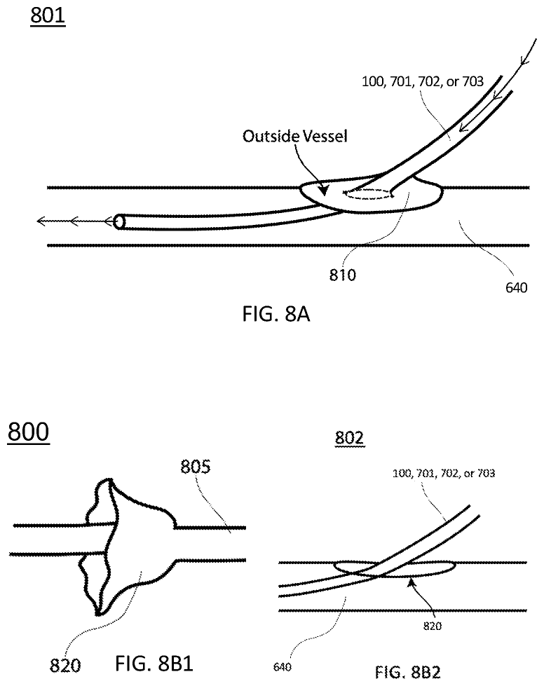

FIGS. 8A and 8B are illustrations of a bidirectional flow cannula 805 inserted in blood vessel 640 and a bleeding mitigation mechanism 810 used at, or near, the bidirectional flow cannula insertion site. In particular FIG. 8A is an illustration of a system for mitigating bleeding from a bidirectional flow cannula insertion site that includes a bidirectional flow cannula like bidirectional flow cannulas 100, 701, 702, or 703 and a bleeding mitigation mechanism 810 positioned outside vessel 640 at the insertion site that acts as a physical barrier for blood escaping from vessel 640. Bleeding mitigation mechanism may be, for example, a patch, bandage, or material adhered to the vessel at the insertion site. Exemplary bleeding mitigation mechanisms 810 include but are not limited to fabric or polymer materials applied to vessel 640 at the insertion site.

FIG. 8B1 is an illustration of an exemplary bidirectional flow cannula 800 that includes a bleeding mitigation mechanism 820 in the form of a skirt that, once positioned within vessel 640 (as shown in FIG. 8B2) may flare outwards to occlude any portions of the insertion cite not occluded by bidirectional flow cannula 850 thereby providing a physical barrier for blood that may otherwise escape from the insertion site and/or vessel 640.

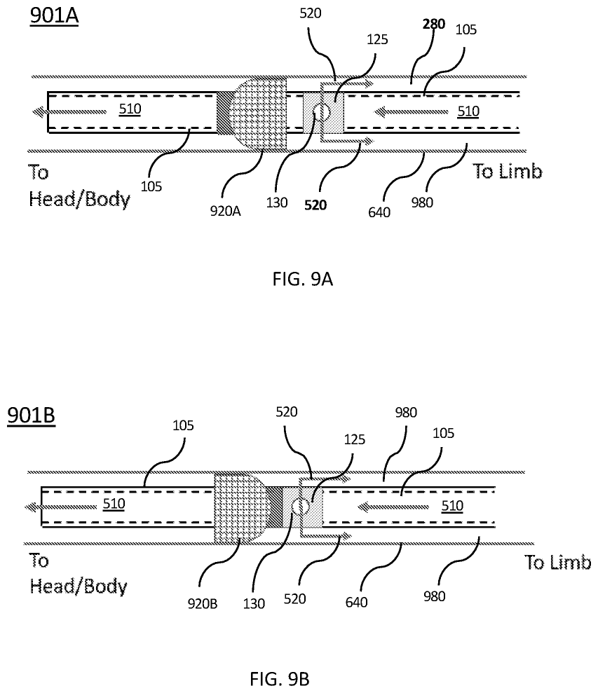

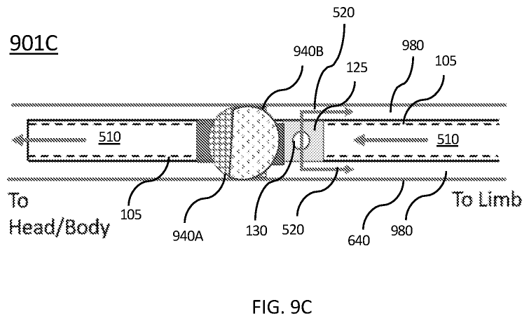

On some occasions, a skirt and/or a flared device may be used with one or more of the bidirectional flow cannulas disclosed herein. The skirt and/or flared device may be configured to, for example, approximate one or more features (e.g., flexibility, shape, and/or size) of a vessel and/or vessel wall which may, at times, enable a backflow of blood to the limb. The skirts may, or may not, be used with a balloon such as balloon 110. Skirts, or flared devices, used with bidirectional flow cannulas may be made from any flexible material (e.g., silicon, fabric, a thin polymer sheet, or plastic) and may be attached to one end of the exterior of main cannula tube 105. FIGS. 9A-9C provide side views of a few exemplary skirts, or flared devices, 920 that may be included with and/or attached to a bidirectional flow cannula like bidirectional flow cannulas 100, 701, 702, or 703 and/or main cannula tube 105. In particular, FIG. 9A is a side view of a first skirted bidirectional flow canula 901A, positioned within a blood vessel 640, and oriented so that a portion of blood vessel 640 extending toward the head or body of a patient is on the left of FIG. 9A and a portion of blood vessel 640 extending toward the limb (e.g., leg or arm) is on the right side of FIG. 9A. First skirted bidirectional flow canula 901A includes main cannula tube 105 with a first skirt 920A affixed thereto and positioned proximate to a port 130. Port 130 may be configured to facilitate secondary blood flow 520 to the patient's limb by allowing blood to flow out of main cannula tube 105 through port 130 and be directed to the limb as shown in FIG. 9A.

Once in position within blood vessel 640, first skirt 920A may be configured to flare out, or open, as shown in FIG. 9A to partially, or fully, occlude blood vessel 640 so that blood exiting port 130 is forced to travel down into the limb. On some occasions, negative pressure created within blood vessel 640 may assist with the deploying, opening, or flaring of first skirt 920A into an open configuration as shown in FIG. 9A. The negative pressure within blood vessel 640 may be caused by, for example, the Bernoulli effect and/or greater blood pressure in the vessel proximal to the body that may be caused by, for example, insertion of first skirted bidirectional flow canula 901A into vessel 640. The back-pressure assistance provided by first skirt 920A may assist with, for example, pushing of secondary blood flow 520 in a reverse direction (as oriented in FIG. 9A) toward the limb as shown in FIG. 9A while the primary blood flow 510 passes through main cannula tube 150 to the patient's head and body.

Second skirted bidirectional flow canula 901B of FIG. 9B is similar to first skirted bidirectional flow canula 901A except that it includes a second skirt 920B, that is oriented in a different direction (e.g., 180 degrees) than first skirt 920A as shown in FIG. 9B. Third skirted bidirectional flow canula 901C of FIG. 9C is similar to first and second skirted bidirectional flow cannulas 901A and 901B except that it includes a third skirt 920C that includes a first component, or skirt, that is oriented in a manner similar to first skirt 920A and a second component, or skirt, that is oriented in a manner similar to second skirt 920B as shown in FIG. 9C. Bidirectional flow cannula 901C may be configured to take advantage of negative pressure within the vessel caused by blood flow to expand in the proximal and distal directions as shown to, for example, isolate proximal ports 130.

The configurations of FIGS. 9B and 9C assist with creating secondary blood flow 520 back to the patient's limb while the primary blood flow 510 passes through main cannula tube 150 to the patient's head and body. Possible advantages to using skirted bidirectional flow cannulas 901A, 901B, and/or 901C are that the skirts provide very gentle apposition to the vessel wall, so it occludes the vessel using the suction/pressure within the blood vessel to create the vessel occlusion without exerting much pressure on the vessel wall. This may serve to reduce a likelihood of damage to the vessel wall caused by bidirectional flow cannulas 901A, 901B, or 901C when in use. Additionally, or alternatively, an advantage of using skirted bidirectional flow cannulas 901A, 901B, and/or 901C may be the creation of a low profile and soft feature (relative to, for example, a balloon) that effectively ‘self-inflates’ to create isolation of the proximal ports using the pressure/blood flow in the vessel which may, for example, decrease a likelihood of damage when inserting/removing skirted bidirectional flow cannulas 901A, 901B, and/or 901C from the vessel.

In some embodiments, full occlusion of a vessel in which a bidirectional flow cannula such as the bidirectional flow cannulas disclosed herein may not be desired so that, for example, blood may flow past an occluding device such as balloon 110 toward a patient's limb in addition to, or in leu of, a secondary blood flow emanating from a port 130 positioned within second region 102 and/or on a distal side of balloon 110. Partial occlusion of a vessel may be accomplished via, for example, partial inflation of an occluding device and/or use of an occluding device that may not sufficiently expand to fully occlude the vessel (e.g., a balloon with an inflated outer diameter smaller than an inner diameter of a vessel into which a bidirectional flow cannula is inserted).

A size of an inflatable occluding device may be set, maintained, regulated and/or changed via, for example, measuring the air and/or liquid pressure within the inflatable occluding device using, for example, a gauge so that the inflatable occluding device is inflated and/or maintained at a desired air pressure. Additionally, or alternatively, a supply of air and/or liquid provided to the inflatable occluding device may be measured so that the amount of air and/or liquid provided to the inflatable occluding device corresponds with a desired level of inflation of the inflatable occluding device, which may, in turn, correspond to the size of the inflatable occluding device when placed within the vessel and/or a degree of occlusion of the vessel achieved via inflating the inflatable occluding device.

The occluding devices and/or balloons disclosed herein may be configured to completely and/or partially occlude a vessel (e.g., blood vessel) into which they are placed. Full occlusion of the vessel may be achieved via, for example, inflating the occluding device so that it comes into contact with (e.g., touches) and/or presses against an interior diameter of the vessel thereby blocking a flow of liquid (e.g., blood) through the vessel. Partial occlusion of the of the vessel may be achieved via, for example, partially inflating an occlusion device so that it partially (e.g., 40-90%) occludes the vessel and, in these cases, the occlusion device may not come into contact with (e.g., touches) an interior diameter of the vessel. This may allow for some liquid to travel past the occlusion device and on through the vessel.

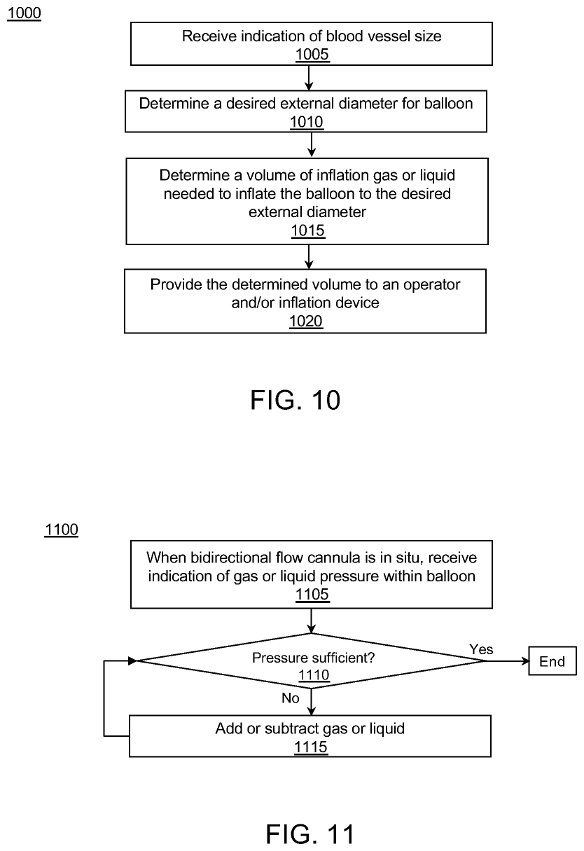

In some instances, a size of the vessel may be known, approximated, and/or measured based on, for example, imaging data (e.g., MRI or ultrasound) and/or approximations correlating to a patient's size, weight, and/or type of vessel into which the bidirectional flow cannula is inserted. When a measured and/or approximated size of a vessel is known, a desired size of the inflatable occluding device may be determined along with a volume of air and/or liquid that may be provided to the inflatable occluding device to achieve the desired size/diameter and/or degree of vessel occlusion when a bidirectional flow cannula including the inflatable occluding device is placed within the vessel. FIGS. 10 and 11 provide flowcharts of exemplary processes 100O and 110O, respectively, for setting parameters for inflation of balloon, like balloon 110, of a bidirectional flow cannula so that the balloon partially occludes a vessel into which the bidirectional flow cannula is inserted. Processes 100O and 110O may be performed singularly or in combination by, for example, a computer or processor that may be in communication with an inflation device (e.g., pump) coupled directly, or indirectly to the balloon and/or a health care provider inserting the bidirectional flow cannula into the patient's vessel. In process 100O, an indication of a size of a blood vessel into which a bidirectional flow cannula is to be inserted may be received (step 1005). The indication may be, for example, an image (e.g., ultrasound, X-ray, CT scan, etc.) from which an internal diameter of the vessel may be measured and/or inferred. A desired external diameter for the balloon may then be determined (step 1010) based on, for example, a desired level of occlusion of the vessel. The determination of step 1010 may be based upon, for example, the indication received in step 1005 and/or a patient characteristic (e.g., health, width of vessel wall, etc.). A volume of inflation gas and/or liquid needed to inflate the balloon to the desired external diameter of step 1010 may then be determined (step 1015) and optionally provided to the balloon (step 1020) via, for example, metered provision of gas and/or liquid to the balloon by the inflation device.

Execution of process 110O may include receiving an indication of gas or liquid pressure within a balloon of a bidirectional flow cannula when the bidirectional flow cannula is in situ within the patient's vessel (step 1105). The indication may be received from, for example, a pressure gauge and/or an individual who received back pressure feedback when pushing the inflation air and/or gas into the balloon. If the pressure is sufficient to achieve partial occlusion (step 1110), process 110O may end. If the pressure is not sufficient to achieve partial occlusion (step 1110), inflation gas and/or liquid may be added or subtracted from the balloon as appropriate (step 1115).

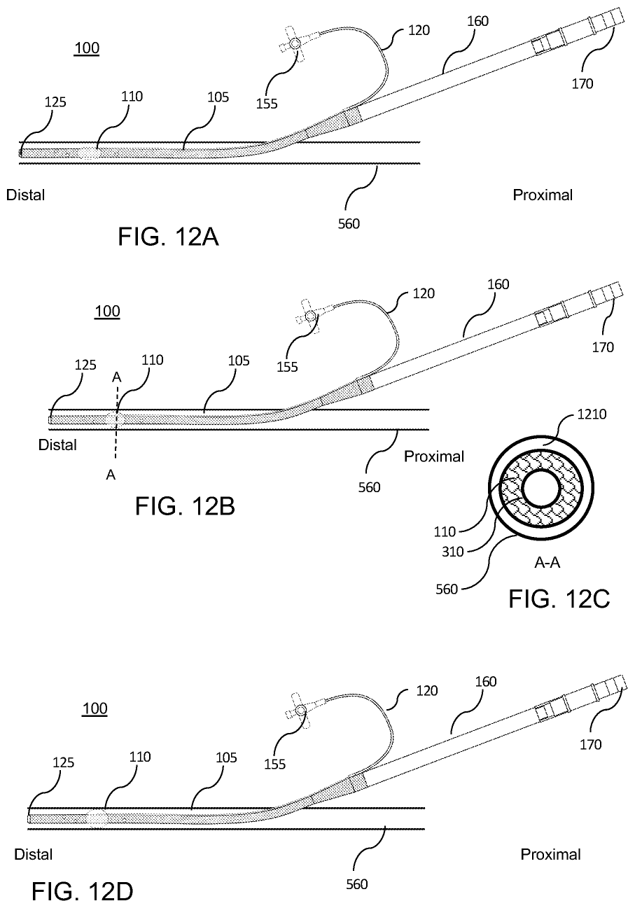

FIGS. 12A, 12B, and 12D show a process for inflation of a balloon 110 of a bidirectional flow cannula like the bidirectional flow cannulas disclosed once the bidirectional flow cannula is situated in vessel 560 and an introducer like introducer 705 has been removed from the bidirectional flow cannula. In particular, FIG. 12A shows bidirectional flow cannula 100 with balloon completely deflated. FIG. 12B shows bidirectional flow cannula 100 with balloon 110 partially (or fully) inflated so that it partially occludes vessel 560 as shown. FIG. 12C is a cross section of vessel 560 when it is partially occluded by balloon 110 and a pathway 1210 for a flow of blood to, for example, a patient's limb is positioned between an exterior surface of balloon 110 and an interior surface of vessel 560. On some occasions (e.g., when partial occlusion of vessel 560 is desired), the deployment process for inflating balloon 110 may end following the process step shown in FIGS. 12B and 12C. On other occasions (e.g., when full occlusion of vessel 560 is desired), balloon 110 may be inflated so that it fully occludes vessel 560 as shown in FIG. 12D.

We claim:

1. A bidirectional flow cannula comprising:
a main cannula tube with a central lumen;
an inflatable balloon circumferentially surrounding a distally positioned portion of an exterior diameter the main tube;
a first plurality of ports positioned within the main cannula tube on a proximal side of the inflatable balloon;
a second plurality of ports positioned within the main cannula tube on a distal side of the inflatable balloon; and
an inflation line in communication with the inflatable balloon and configured to facilitate inflation of the inflatable balloon.

2. The bidirectional flow cannula of claim 1, wherein an interior diameter of the second plurality of ports is smaller than an interior diameter of the first plurality of ports.

3. The bidirectional flow cannula of claim 1, wherein an interior diameter of the second plurality of ports is larger than an interior diameter of the first plurality of ports.

4. The bidirectional flow cannula of any of claims 1-3, wherein the bidirectional flow cannula is configured for use within a patient's femoral artery.

5. The bidirectional flow cannula of any of claims 1-3, wherein the bidirectional flow cannula is configured for use within a patient's femoral vein.

6. The bidirectional flow cannula of any of claims 1-5, further comprising:
a radio-opaque marker positioned on the distal side of the main cannula tube.

7. The bidirectional flow cannula of any of claims 1-6, wherein the inflatable balloon is configured so that it does not fully occlude a patient's blood vessel when positioned therein and inflated.

8. The bidirectional flow cannula of any of claims 1-7, wherein the inflatable balloon is configured so that it fully occludes a patient's blood vessel when positioned therein and inflated.

9. The bidirectional flow cannula of any of claims 1-8, wherein the inflatable balloon has a non-circular cross-section, the cross-section being perpendicular to the main cannula tube.

10. The bidirectional flow cannula of any of claims 1-9, wherein the main cannula tube has a non-circular cross section.

11. The bidirectional flow cannula of any of claims 1-10, wherein the inflation line is resident within a sidewall of the main cannula tube.

12. The bidirectional flow cannula of any of claims 1-11, further comprising:
a proximal basket positioned within a sidewall of the proximal side of the main cannula tube, the proximal basket including a first plurality of holes sized, positioned, and configured, to align with the first plurality of ports.

13. The bidirectional flow cannula of any of claims 1-12, further comprising:
a distal basket positioned within a sidewall of the distal side of the main cannula tube, the distal basket including a second plurality of holes sized, positioned, and configured, to align with the second plurality of ports.

14. The bidirectional flow cannula of any of claims 1-13, wherein the inflation line has a non-circular cross-section, the cross-section being perpendicular to the main cannula tube.

15. The bidirectional flow cannula of any of claims 1-13, wherein the inflation line is positioned on an exterior surface of the main cannula tube, the bidirectional flow cannula further comprising:
a set of supports positioned at a junction of the inflation line and the main cannula tube.

16. The bidirectional flow cannula of any of claims 1-13, wherein the inflation line is positioned within a sidewall of the main cannula tube.

17. The bidirectional flow cannula of any of claims 1-16, wherein a port of at least one of the first plurality of ports and the second plurality of ports is oriented at an angle that is not perpendicular to the main cannula tube.

18. The bidirectional flow cannula of any of claims 1-17, wherein a quantity of ports included in the first plurality of ports is larger than a quantity of ports included in the second plurality of ports.

19. The bidirectional flow cannula of claim 18, wherein the first plurality of ports includes eight ports, and the second plurality of ports includes two ports.

20. The bidirectional flow cannula of any of claims 1-17, wherein a quantity of ports included in the second plurality of ports is larger than a quantity of ports included in the first plurality of ports.

21. The bidirectional flow cannula of any of claims 1-20, wherein the first plurality of ports includes 1-14 ports.

22. The bidirectional flow cannula of any of claims 1-21, wherein the second plurality of ports includes 1-14 ports.

23. A method for using a bidirectional flow cannula comprising:
inserting a distal portion of the bidirectional flow cannula into a blood vessel in a patient's limb, the distal portion of the bidirectional flow cannula including an inflatable balloon;
once in situ in the patient's blood vessel, inflating the inflatable balloon to a volume that partially occludes the patient's blood vessel;
supplying a volume of blood to the bidirectional flow cannula so that the volume of blood is injected into the patient's blood vessel via a tip of the bidirectional flow cannula positioned on a distal end of the bidirectional flow cannula, wherein a first portion of the volume of blood supplied to the bidirectional flow cannula flows away from the bidirectional flow cannula along the patient's blood vessel toward the patient's heart and a second portion of the volume of blood supplied to the bidirectional flow cannula flows toward, and past, the inflatable balloon toward the patient's limb.

24. The method of claim 23, wherein the inflatable balloon is inflated to a predetermined level of pressure.

25. The method of claim 23, wherein the inflatable balloon is inflated with a predetermined volume of gas.

26. The method of any of claims 23-25, wherein the inflatable balloon is inflated with a fluid.

27. The method of any of claims 23-26, further comprising:
receiving an indication of a size of the blood vessel prior to insertion of the bidirectional flow cannula into the blood vessel, a volume of gas provided to the inflatable balloon being responsive to the received indication.

28. The method of any of claims 23-27, further comprising:
receiving an indication of a size of the blood vessel prior to insertion of the bidirectional flow cannula into the blood vessel, a volume of liquid provided to the inflatable balloon being responsive to the received indication.

29. The method of any of claims 23-28, wherein the bidirectional flow cannula further includes a plurality of ports positioned on a proximal side of the inflatable balloon, wherein a third portion of the volume of blood supplied to the bidirectional flow cannula flows from the ports and toward the patient's limb.

30. The method of claim 23-29, wherein the bidirectional flow cannula further includes a plurality of ports positioned on a distal side of the inflatable balloon, wherein a fourth portion of the volume of blood supplied to the bidirectional flow cannula flows from the ports positioned on a distal side of the inflatable balloon and toward the patient's heart, thereby reducing at least one of a velocity and a pressure of the first portion of blood exiting the tip of the bidirectional flow cannula.

31. A method for using a bidirectional flow cannula comprising:
inserting a distal portion of the bidirectional flow cannula into a blood vessel in a patient's limb, the distal portion of the bidirectional flow cannula including an inflatable balloon and a plurality of ports positioned on a proximal side of the inflatable balloon;
once in situ in the patient's blood vessel, inflating the inflatable balloon to a volume that occludes the patient's blood vessel;
supplying a volume of blood to the bidirectional flow cannula so that the volume of blood is injected into the patient's blood vessel via a tip of the bidirectional flow cannula positioned on a distal end of the bidirectional flow cannula, wherein a first portion of the volume of blood supplied to the bidirectional flow cannula flows away from the bidirectional flow cannula along the patient's blood vessel toward the patient's heart and a second portion of the volume of blood supplied to the bidirectional flow cannula flows from the plurality of ports toward the patient's limb.

32. The method of claim 31, wherein the bidirectional flow cannula further includes a plurality of ports positioned on a distal side of the inflatable balloon, wherein a fourth portion of the volume of blood supplied to the bidirectional flow cannula flows from the ports positioned on a distal side of the inflatable balloon and toward the patient's heart, thereby reducing at least one of a velocity and a pressure of the first portion of blood exiting the tip of the bidirectional flow cannula.
